SEQUENCE LISTING (10 sequences)

SEQ ID NO: 1 - DNA, 18 bp, Artificial Sequence
  Description of Artificial Sequence Synthetic oligonucleotide
gcgtgcctcctcactggc

SEQ ID NO: 2 - DNA, 18 bp, Artificial Sequence
  Description of Artificial Sequence Synthetic oligonucleotide
gcgcgcctcctcgctggc

SEQ ID NO: 3 - DNA, 18 bp, Artificial Sequence
  Description of Artificial Sequence Synthetic oligonucleotide
gcatgcttccacacaggc

SEQ ID NO: 4 - DNA, 18 bp, Artificial Sequence
  Description of Combined DNA/RNA Molecule Synthetic oligonucleotide
gcgugcctcctcacuggc

SEQ ID NO: 5 - DNA, 18 bp, Artificial Sequence
  Description of Combined DNA/RNA Molecule Synthetic oligonucleotide
gcgcgcctcctcgcuggc

SEQ ID NO: 6 - DNA, 18 bp, Artificial Sequence
  Description of Combined DNA/RNA Molecule Synthetic oligonucleotide
gcgtgccuccucactggc

SEQ ID NO: 7 - DNA, 18 bp, Artificial Sequence
  Description of Combined DNA/RNA Molecule Synthetic oligonucleotide
gcgcgccuccucgctggc

SEQ ID NO: 8 - DNA, 18 bp, Artificial Sequence
  Description of Combined DNA/RNA Molecule Synthetic oligonucleotide
gcatgcauccgcacaggc

SEQ ID NO: 9 - DNA, 18 bp, Artificial Sequence
  Description of Combined DNA/RNA Molecule Synthetic oligonucleotide
gcaugcttccacacaggc

SEQ ID NO: 10 - DNA, 18 bp, Artificial Sequence
  Description of Combined DNA/RNA Molecule Synthetic oligonucleotide
gcaugcatccgcacaggc

(19) United States
(12) Patent Application Publication
(10) Pub. No.: US 2005/0054600 A1
(43) Pub. Date: Mar. 10, 2005

(54) Modified protein kinase a-specific oligonucleotides and methods of their use

(75) Inventor: Sudhir Agrawal, Shrewsbury, MA (US)

Correspondence Address: WILMER CUTLER PICKERING HALE AND DORR LLP, 60 STATE STREET, BOSTON, MA, 02109, US

(73) Assignee: Hybridon, Inc., Cambridge, MA (US)

(21) Appl. No.: 10/854,989

(22) Filed: May 27, 2004

Related U.S. Application Data
(63) Continuation-in-part of application No. 09/412,947, filed on Oct. 5, 1999.
(63) Continuation-in-part of application No. 09/708,786, filed on Nov. 8, 2000.
(63) Continuation-in-part of application No. 09/022,965, filed on Feb. 12, 1998, now Pat. No. 6,624,293.
(63) Continuation-in-part of application No. 08/532,979, filed on Sep. 22, 1995, now Pat. No. 5,969,117.
Application No. 08/532,979 is a continuation-in-part of application No. 08/516,454, filed on Aug. 17, 1995, now Pat. No. 5,652,356.
(60) Provisional application No. 60/164,182, filed on Nov. 9, 1999.
(60) Provisional application No. 60/103,098, filed on Oct. 5, 1998.
(60) Provisional application No. 60/040,740, filed on Mar. 12, 1997.

(51) Int. Cl. A61K 48/00

(52) U.S. Cl. 514/44

(57) ABSTRACT

The present invention relates to pharmaceutical compositions and methods for inhibiting the proliferation of cancer cells or treating cancer in an afflicted subject. The invention utilizes modified oligonucleotides complementary to nucleic acid encoding protein kinase A subunit RIα.

### CROSS-REFERENCE TO RELATED APPLICATIONS

This application is a continuation-in-part of U.S. Ser. No. 09/412,947, filed Oct. 5, 1999 and U.S. Ser. No. 09/708,786, filed Nov. 8, 2000. U.S. Ser. No. 09/708,786 claims the benefit of U.S. Ser. No. 60/164,182, filed Nov. 9, 1999. U.S. Ser. No. 09/412,947 claims the benefit of U.S. Ser. No. 60/103,098, filed on Oct. 5, 1998, and is a continuation-in-part of U.S. Ser. No. 09/022,965, filed on Feb. 12, 1998, (now U.S. Pat. No. 6,624,293), U.S. Ser. No. 09/022,965 claims the benefit U.S. Ser. No. 60/040,740, filed on Mar. 12, 1997, and is a continuation-in-part of U.S. Ser. No. 08/532,979, filed Sep. 22, 1995, (now U.S. Pat. No. 5,969,117), which is a continuation-in-part of U.S. Ser. No. 08/516,454, filed Aug. 17, 1995 (now U.S. Pat. No. 5,652,356). The issued U.S. patents, applications, published foreign applications, and references cited herein are hereby incorporated by reference.

### FIELD OF THE INVENTION

The present invention relates to cancer therapy. More specifically, the present invention relates to the inhibition of the proliferation of cancer cells using modified antisense oligonucleotides complementary to nucleic acid encoding the protein kinase A RIα subunit in combination with other anticancer agents.

### BACKGROUND OF THE INVENTION

The development of effective cancer therapies has been a major focus of biomedical research. Surgical procedures have been developed and used to treat patients whose tumors are confined to particular anatomical sites. However, at present, only about 25% of patients have tumors that are truly confined and amenable to surgical treatment alone (Slapak et al. in Harrison's Principles of Internal Medicine (Isselbacher et al., eds.) McGraw-Hill, Inc., NY (1994) pp. 1826-1850). Radiation therapy, like surgery, is a local modality whose usefulness in the treatment of cancer depends to a large extent on the inherent radiosensitivity of the tumor and its adjacent normal tissues. However, radiation therapy is associated with both acute toxicity and long term sequelae. Furthermore, radiation therapy is known to be mutagenic, carcinogenic, and teratogenic (Slapak et al., ibid.).

Systemic chemotherapy alone or in combination with surgery and/or radiation therapy is currently the primary treatment available for disseminated malignancies. However, conventional chemotherapeutic agents which either block enzymatic pathways or randomly interact with DNA irrespective of the cell phenotype, lack specificity for killing neoplastic cells. Thus, systemic toxicity often results from standard cytotoxic chemotherapy.

Furthermore, many potential pharmaceutical agents fail to be used therapeutically due to excessive toxicity or limited bioavailability. In some instances, these limiting factors can be ameliorated by modifying the pharmaceutical agent to create a prodrug. The prodrug is then converted by the body into the pharmaceutically active substance. For example, International Appln. No. PCT/US97/14751 discloses the manufacture of oligonucleotide prodrugs having ester or amide modifications that cover a non-bridging oxygen of the phosphodiester linkage. Kuhn (Oncol., Supplement No. 6, 39-42 (1998)) discloses that CPT-11 (Camptosar®) is an antineoplastic prodrug that is converted by carboxylesterase activity in the liver and other tissues to the active agent SN-38. Cerosimo (Ann. Phannacother. 32: 1324-1333 (1998)) teaches that the parent compound of CPT-11, camptothecin, was unable to be developed as a pharmaceutical due to severe toxicity.

Due to the presence of carboxylesterases and amidases in the liver and other tissues, the ability to make prodrugs which have added ester or amide groups is a generalizable phenomenon. However, these compounds generally retain at least some of the toxicity of the parent compound, due to rapid hydrolysis of the prodrug. Kuhn, supra, discloses that SN-38, the active metabolite of CPT-11 still causes diarrhea, which is the limiting toxicity of the parent compound, camptothecin.

Thus, there is a need for methods to administer prodrugs in a manner that maximizes their efficacy while avoiding significant toxicity. Ideally, such methods should affect the manner in which the body processes prodrugs, and thus would be applicable to a broad range of prodrugs.

In addition, the development of agents that block replication, transcription, or translation in transformed cells, and at the same time defeat the ability of cells to become resistant, has been the goal of many approaches to chemotherapy.

One strategy is to down-regulate the expression of a gene associated with the neoplastic phenotype in a cell. A technique for turning off a single activated gene is the use of antisense oligodeoxynucleotides and their analogues for inhibition of gene expression (Zamecnik et al. (1978) Proc. Natl. Acad. Sci. (USA) 75:280-284). An antisense oligonucleotide targeted at a gene involved in the neoplastic cell growth should specifically interfere only with the expression of that gene, resulting in arrest of cancer cell growth. The ability to specifically block or down-regulate expression of such genes provides a powerful tool to explore the molecular basis of normal growth regulation, as well as the opportunity for therapeutic intervention (see, e.g., Cho-Chung (1993) Curr. Opin. Thera. Patents 3:1737-1750). The identification of genes that confer a growth advantage to neoplastic cells as well as other genes causally related to cancer and the understanding of the genetic mechanism(s) responsible for their activation makes the antisense approach to cancer treatment possible.

One such gene encodes the RIα subunit of cyclic AMP (cAMP)-dependent protein kinase A (PKA) (Krebs (1972) Curr. Topics Cell. Regul. 5:99-133). Protein kinase is bound by cAMP, which is thought to have a role in the control of cell proliferation and differentiation (see, e.g., Cho-Chung (1980) J. Cyclic Nucleotide Res. 6:163-167). There are two types of PKA, type I (PKA-I) and type II (PKA-II), both of which share a common C subunit but each containing distinct R subunits, RI and RII, respectively (Beebe et al. in The Enzymes: Control by Phosphorylation, 17(A):43-111 (Academic, New York, 1986). The R subunit isoforms differ in tissue distribution (Øyen et al. (1988) FEBS Lett. 229:391-394; Clegg et al. (1988) Proc. Natl. Acad. Sci. (USA) 85:3703-3707) and in biochemical properties (Beebe et al. in The Enzymes: Control by Phosphorylation, 17(A):43-111 (Academic Press, NY, 1986); Cadd et al. (1990) J. Biol. Chem. 265:19502-19506). The two general isoforms of the R subunit also differ in their subcellular localization: RI is found throughout the cytoplasm; whereas RII localizes to nuclei, nucleoli, Golgi apparatus and the microtubule organizing center (see, e.g., Lohmann in Advances in Cyclic Nucleotide and Protein Phosphorylation Research, 18:63-117 (Raven, New York, 1984; and Nigg et al. (1985) Cell 41:1039-1051).

An increase in the level of RIα expression has been demonstrated in human cancer cell lines and in primary tumors, as compared with normal counterparts, in cells after transformation with the Ki-ras oncogene or transforming growth factor-α, and upon stimulation of cell growth with granulocyte-macrophage colony-stimulating factor (GM-CSF) or phorbol esters (Lohmann in Advances in Cyclic Nucleotide and Protein Phosphorylation Research, 18:63-117 (Raven, New York, 1984); and Cho-Chung (1990) Cancer Res. 50:7093-7100). Conversely, a decrease in the expression of RIα has been correlated with growth inhibition induced by site-selective cAMP analogs in a broad spectrum of human cancer cell lines (Cho-Chung (1990) Cancer Res. 50:7093-7100). It has also been determined that the expression of RI/PKA-I and RII/PKA-II has an inverse relationship during ontogenic development and cell differentiation (Lohmann in Advances in Cyclic Nucleotide and Protein Phosphorylation Research, Vol. 18, 63-117 (Raven, New York, 1984); Cho-Chung (1990) Cancer Res. 50:7093-7100). The RIα subunit of PKA has thus been hypothesized to be an ontogenic growth-inducing protein whose constitutive expression disrupts normal ontogenic processes, resulting in a pathogenic outgrowth, such as malignancy (Nesterova et al. (1995) Nature Med. 1:528-533).

Antisense oligonucleotides directed to the RIα gene have been prepared. U.S. Pat. No. 5,271,941 describes phosphodiester-linked antisense oligonucleotides complementary to a region of the first 100 N-terminal amino acids of RIα which inhibit the expression of RIα in leukemia cells in vitro. In addition, antisense phosphorothioate oligodeoxynucleotides corresponding to the N-terminal 8-13 codons of the RIα gene was found to reduce in vivo tumor growth in nude mice (Nesterova et al. (1995) Nature Med. 1:528-533).

Unfortunately, problems have been encountered with the use of phosphodiester-linked (PO) oligonucleotides and some phosphorothioate-linked (PS) oligonucleotides. It is known that nucleases in the serum readily degrade PO oligonucleotides. Replacement of the phosphodiester internucleotide linkages with phosphorothioate internucleotide linkages has been shown to stabilize oligonucleotides in cells, cell extracts, serum, and other nuclease-containing solutions (see, e.g., Bacon et al. (1990) Biochem. Biophys. Meth. 20:259) as well as in vivo (Iversen (1993) Antisense Research and Application (Crooke, ed) CRC Press, 461). However, some PS oligonucleotides have been found to exhibit an immunostimulatory response, which in certain cases may be undesirable. For example, Galbraith et al. (Antisense Res. & Dev. (1994) 4:201-206) disclose complement activation by some PS oligonucleotides. Henry et al. (Pharm. Res. (1994)11:PPDM8082) disclose that some PS oligonucleotides may potentially interfere with blood clotting.

There is, therefore, a need for modified oligonucleotides directed to cancer-related genes that retain gene expression inhibition properties while producing fewer side effects than conventional oligonucleotides.

### SUMMARY OF THE INVENTION

The present invention relates to modified oligonucleotides useful for studies of gene expression and for the antisense therapeutic approach. In some embodiments the invention utilizes modified oligonucleotides that down-regulate the expression of the RIα gene while producing fewer side effects than conventional oligonucleotides. In various embodiments the invention utilizes modified oligonucleotides that demonstrate reduced mitogenicity, reduced activation of complement and reduced antithrombotic properties, relative to conventional oligonucleotides.

It is known that some phosphorothioate (PS) oligonucleotides cause an immunostimulatory response in subjects to whom they have been administered, which may be undesirable in some cases. It is also known that exclusively phosphodiester—or exclusively phosphorothioate-linked oligonucleotides directed to the first 100 nucleotides of the RIα nucleic acid inhibit cell proliferation.

It has now been discovered that modified oligonucleotides complementary to the protein kinase A RIα subunit gene inhibit the growth of tumors in vivo with at least the activity of a comparable PO- or PS-linked oligonucleotide with fewer side effects. It has also been determined that modified oligonucleotides complementary to the protein kinase A RIα subunit gene have a synergistic growth inhibitory effect with antibodies that bind to epidermal growth factor receptor (EGFR) or with various classes of cytotoxic drugs, including taxanes, platinum-derived agents, topoisomerase II-selective drugs, and topoisomerase I inhibitors. These classes of cytotoxic drugs can include prodrugs as well, including but not limited to, esters or amides of anti-cancer drugs, such as topoisomerase I inhibitors.

These findings have been exploited to produce the present invention, which includes synthetic hybrid, inverted hybrid, and inverted chimeric oligonucleotides and compositions of matter for specifically down-regulating protein kinase A subunit RIα gene expression with reduced side effects. Such inhibition of gene expression is useful as an alternative to mutant analysis for determining the biological function and role of protein kinase A-related genes in cell proliferation and tumor growth. Such inhibition of RIα gene expression can also be used to therapeutically treat diseases and disorders that are caused by the over-expression or inappropriate expression of the gene.

In a first aspect, the invention provides a method for inhibiting proliferation of cancer cells. This method comprises administering to the cells a first therapeutic agent and a second therapeutic agent. The first therapeutic agent comprises a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises a topoisomerase I inhibitor. The administering steps may be performed simultaneously or sequentially in any order.

As used herein, the term “synthetic oligonucleotide” includes chemically synthesized polymers of three up to 50. In some embodiments the synthetic oligonucleotide is from about 15 to about 30. In certain embodiments the synthetic oligonucleotide is 18 ribonucleotide and/or deoxyribonucleotide monomers connected together or linked by at least one, and in some embodiments more than one, 5′ to 3′ internucleotide linkage.

For purposes of the invention, the terms “oligonucleotide sequence that is complementary to a genomic region or an RNA molecule transcribed therefrom” and “oligonucleotide complementary to” are intended to mean an oligonucleotide that binds to the target nucleic acid sequence under physiological conditions, e.g., by Watson-Crick base pairing (interaction between oligonucleotide and single-stranded nucleic acid) or by Hoogsteen base pairing (interaction between oligonucleotide and double-stranded nucleic acid) or by any other means including in the case of an oligonucleotide binding to RNA, causing pseudoknot formation. Binding by Watson-Crick or Hoogsteen base pairing under physiological conditions is measured as a practical matter by observing interference with the function of the nucleic acid sequence.

In some embodiments, the oligonucleotide is a hybrid oligonucleotide, which in certain embodiments has a nucleotide sequence consisting of the nucleotide sequence set forth in SEQ ID NO:4. In other embodiments, the oligonucleotide is an inverted hybrid oligonucleotide, which in certain embodiments has a nucleotide sequence consisting of the nucleotide sequence set forth in SEQ ID NO:6. In other embodiments, the oligonucleotide is an inverted chimeric oligonucleotide, which in certain embodiments has a nucleotide sequence consisting of the nucleotide sequence set forth in SEQ ID NO:1.

In some embodiments, the oligonucleotide further comprises a 2′-O-substituted nucleotide. For purposes of the invention, the term “2′-O-substituted” means substitution of the 2′ position of the pentose moiety with an —O— lower alkyl group containing 1-6 saturated or unsaturated carbon atoms, or with an —O-aryl or allyl group having 2-6 carbon atoms, wherein such alkyl, aryl or allyl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group, but not with a 2′-H group. In some embodiments, each of the 3′ and 5′ flanking ribonucleotide regions of an oligonucleotide of the invention comprises at least one 2′-O-alkyl substituted ribonucleotide. In one useful embodiment, the 2′-O-alkyl-substituted nucleotide is a 2′-O-methyl ribonucleotide. In other useful embodiments, the 3′ and 5′ flanking ribonucleotide regions of an oligonucleotide of the invention comprise at least four 2′-O-methyl ribonucleotides. In some embodiments, the ribonucleotides and deoxyribonucleotides of the hybrid oligonucleotide are linked by phosphorothioate internucleotide linkages. In some embodiments, this phosphorothioate region or regions have from about four to about 18 nucleosides joined to each other by 5′ to 3′ phosphorothioate linkages. In some embodiments, this phosphorothioate region or regions have from about 5 to about 18 such phosphorothioate-linked nucleosides. The phosphorothioate linkages may be mixed Rₚ and Sₚ enantiomers, or they may be stereoregular or substantially stereoregular in either Rₚ or Sₚ form (see Iyer et al. (1995) Tetrahedron Asymmetry 6:1051-1054).

In certain embodiments, the second therapeutic agent is administered prior to administration of the first therapeutic agent.

In some embodiments, the cancer cells are human cancer cells, which in some embodiments are selected from the group consisting of breast cancer cells, colon cancer cells, and ovarian cancer cells.

In some embodiments the topoisomerase I inhibitor is CPT-11.

In a second aspect the invention provides a pharmaceutical composition comprising a first therapeutic agent and a second therapeutic agent. The first therapeutic agent comprises a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises a topoisomerase I inhibitor.

In some embodiments, the oligonucleotide is a hybrid oligonucleotide, which in certain embodiments has a nucleotide sequence consisting of the nucleotide sequence set forth in SEQ ID NO:4. In other embodiments, the oligonucleotide is an inverted hybrid oligonucleotide, which in certain embodiments has a nucleotide sequence consisting of the nucleotide sequence set forth in SEQ ID NO:6. In other embodiments, the oligonucleotide is an inverted chimeric oligonucleotide, which in certain embodiments has a nucleotide sequence consisting of the nucleotide sequence set forth in SEQ ID NO:1. In some embodiments, the oligonucleotide further comprises a 2′-O-substituted nucleotide.

In some embodiments the topoisomerase I inhibitor is CPT-11.

In a third aspect, the invention provides a method for treating cancer in an afflicted subject. This method comprises administering to the subject a first therapeutic agent and a second therapeutic agent. The first therapeutic agent comprises a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises a topoisomerase I inhibitor. The administering steps may be performed simultaneously or sequentially in any order.

In certain embodiments, the second therapeutic agent is administered prior to administration of the first therapeutic agent.

In some embodiments, the subject is a human. In certain embodiments the human has a cancer selected from the group consisting of breast cancer, colon cancer, and ovarian cancer.

In some embodiments, the oligonucleotide is a hybrid oligonucleotide, which in certain embodiments has a nucleotide sequence consisting of the nucleotide sequence set forth in SEQ ID NO:4. In other embodiments, the oligonucleotide is an inverted hybrid oligonucleotide, which in certain embodiments has a nucleotide sequence consisting of the nucleotide sequence set forth in SEQ ID NO:6. In other embodiments, the oligonucleotide is an inverted chimeric oligonucleotide, which in certain embodiments has a nucleotide sequence consisting of the nucleotide sequence set forth in SEQ ID NO:1. In some embodiments, the oligonucleotide further comprises a 2′-O-substituted nucleotide.

In some embodiments the topoisomerase I inhibitor is CPT-11.

In another aspect, the invention provides a method for inhibiting proliferation of cancer cells. The method comprises administering to the cells a first therapeutic agent and a second therapeutic agent. The first therapeutic agent comprises a synthetic, modified oligonucleotide complementary to at least 15 consecutive nucleotides of the nucleic acid encoding the N-terminal 8-13 codons of protein kinase A subunit RIα wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises a topoisomerase I inhibitor. The administering steps may be performed simultaneously or sequentially in any order.

In another aspect, the invention provides a pharmaceutical composition comprising a first therapeutic agent and a second therapeutic agent. The first therapeutic agent comprises a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises a topoisomerase I inhibitor.

In yet another aspect, the invention provides a method for treating cancer in an afflicted subject. The method comprises administering to the subject a first therapeutic agent and a second therapeutic agent. The first therapeutic agent comprises a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises a topoisomerase I inhibitor. The administering steps may be performed simultaneously or sequentially in any order.

In another aspect, the invention provides a method for inhibiting proliferation of cancer cells. This method comprises administering to the cells a first therapeutic agent and a second therapeutic agent. The first therapeutic agent comprises a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises CPT-11. The administering steps may be performed simultaneously or sequentially in any order.

In some embodiments, the oligonucleotide is a hybrid oligonucleotide, which in certain embodiments has a nucleotide sequence consisting of the nucleotide sequence set forth in SEQ ID NO:4. In other embodiments, the oligonucleotide is an inverted hybrid oligonucleotide, which in certain embodiments has a nucleotide sequence consisting of the nucleotide sequence set forth in SEQ ID NO:6. In other embodiments, the oligonucleotide is an inverted chimeric oligonucleotide, which in certain embodiments has a nucleotide sequence consisting of the nucleotide sequence set forth in SEQ ID NO:1. In some embodiments, the oligonucleotide further comprises a 2′-O-substituted nucleotide.

In certain embodiments, the second therapeutic agent is administered prior to administration of the first therapeutic agent.

In some embodiments, the cancer cells are human cancer cells, which in some embodiments are selected from the group consisting of breast cancer cells, colon cancer cells, and ovarian cancer cells.

In yet another aspect the invention provides a pharmaceutical composition comprising a first therapeutic agent and a second therapeutic agent. The first therapeutic agent comprises a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises CPT-11.

In some embodiments, the oligonucleotide is a hybrid oligonucleotide, which in certain embodiments has a nucleotide sequence consisting of the nucleotide sequence set forth in SEQ ID NO:4. In other embodiments, the oligonucleotide is an inverted hybrid oligonucleotide, which in certain embodiments has a nucleotide sequence consisting of the nucleotide sequence set forth in SEQ ID NO:6. In other embodiments, the oligonucleotide is an inverted chimeric oligonucleotide, which in certain embodiments has a nucleotide sequence consisting of the nucleotide sequence set forth in SEQ ID NO:1. In some embodiments, the oligonucleotide further comprises a 2′-O-substituted nucleotide.

In another aspect, the invention provides a method for treating cancer in an afflicted subject. This method comprises administering to the subject a first therapeutic agent and a second therapeutic agent. The first therapeutic agent comprises a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises CPT-11. The administering steps may be performed simultaneously or sequentially in any order.

In certain embodiments, the second therapeutic agent is administered prior to administration of the first therapeutic agent.

In some embodiments, the subject is a human. In certain embodiments the human has a cancer selected from the group consisting of breast cancer, colon cancer, and ovarian cancer.

In some embodiments, the oligonucleotide is a hybrid oligonucleotide, which in certain embodiments has a nucleotide sequence consisting of the nucleotide sequence set forth in SEQ ID NO:4. In other embodiments, the oligonucleotide is an inverted hybrid oligonucleotide, which in certain embodiments has a nucleotide sequence consisting of the nucleotide sequence set forth in SEQ ID NO:6. In other embodiments, the oligonucleotide is an inverted chimeric oligonucleotide, which in certain embodiments has a nucleotide sequence consisting of the nucleotide sequence set forth in SEQ ID NO:1. In some embodiments, the oligonucleotide further comprises a 2′-O-substituted nucleotide.

In another aspect, the invention provides a method for inhibiting proliferation of cancer cells. The method comprises administering to the cells a first therapeutic agent and a second therapeutic agent. The first therapeutic agent comprises a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises CPT-11. The administering steps may be performed simultaneously or sequentially in any order.

In another aspect, the invention provides pharmaceutical composition comprising a first therapeutic agent and a second therapeutic agent. The first therapeutic agent comprises a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises CPT-11.

In yet another aspect, the invention provides a method for treating cancer in an afflicted subject. The method comprises administering to the subject a first therapeutic agent and a second therapeutic agent. The first therapeutic agent comprises a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide. The hybrid oligonucleotide comprises a region of at least two deoxyribonucleotides, flanked by 3′ and 5′ flanking ribonucleotide regions each having at least four ribonucleotides. The inverted hybrid oligonucleotide comprises a region of at least four ribonucleotides flanked by 3′ and 5′ flanking deoxyribonucleotide regions of at least two deoxyribonucleotides, and the inverted chimeric oligonucleotide comprises an oligonucleotide nonionic region of at least four nucleotides flanked by two oligonucleotide phosphorothioate regions. The second therapeutic agent comprises CPT-11. The administering steps may be performed simultaneously or sequentially in any order.

In another aspect, the invention provides a method for inhibiting proliferation of cancer cells. The method comprises administering to the cells a first therapeutic agent comprising a synthetic, modified oligonucleotide comprising SEQ ID NO:4 and administering to the cells a second therapeutic agent comprising CPT-11. The oligonucleotide has four 2′-O-methyl ribonucleotides at the 3′ terminus and at the 5′ terminus and phosphorothioate internucleotide linkages between every nucleoside. The administering steps may be performed simultaneously or sequentially in any order.

In another aspect, the invention provides a pharmaceutical composition comprising a first therapeutic agent comprising a synthetic, modified oligonucleotide comprising SEQ ID NO:4 and a second therapeutic agent comprising CPT-11. The oligonucleotide has four 2′-O-methyl ribonucleotides at the 3′ terminus and at the 5′ terminus and phosphorothioate internucleotide linkages between every nucleoside.

In yet another aspect, the invention provides a method for treating cancer in an afflicted subject. The method comprises administering to the subject a first therapeutic agent comprising a synthetic, modified oligonucleotide comprising SEQ ID NO:4 and administering to the subject a second therapeutic agent comprising CPT-11. The oligonucleotide has four 2′-O-methyl ribonucleotides at the 3′ terminus and at the 5′ terminus and phosphorothioate internucleotide linkages between every nucleoside. The administering steps may be performed simultaneously or sequentially in any order.

### BRIEF DESCRIPTION OF THE DRAWINGS

The present invention and the various features thereof may be more fully understood from the following description, when read together with the accompanying drawings in which:

FIG. 1 is a graphic representation showing the effect of modified oligonucleotides utilized according to various embodiments of the invention on tumor size in a mouse relative to various controls.

FIG. 2 is a graphic representation showing the effect of HYB 165 with docetaxel and monoclonal antibody MAb C225 on the growth of ZR-75-1 human breast cancer cells.

FIG. 3 is a graphic representation showing the effect of HYB 508 with docetaxel and monoclonal antibody MAb C225 on the growth of ZR-75-1 human breast cancer cells.

FIG. 4 is a graphic representation showing the effect of HYB 165 with or without paclitaxel on the growth of geo human colon cancer cells.

FIG. 5 is a graphic representation showing the effect of HYB 165 and its control HYB 508 on the growth of 1A9PTX22 human ovarian cancer cells.

FIG. 6 is a graphic representation showing the effect of HYB 165 and its control HYB 508 on the growth of 1A9PTX10 human ovarian cancer cells.

FIG. 7 is a graphic representation showing the effect of HYB 165 and its control HYB 508 on the growth of 1A9 human ovarian cancer cells.

FIG. 8 is a graphic representation showing the effect of HYB 508 with or without monoclonal antibody MAb C225 on the growth of ZR-75-1 human breast cancer cells.

FIG. 9 is a graphic representation showing the effect of HYB 165 and HYB 618 on the growth of OVCAR-3 ovarian cancer cells.

FIG. 10 is a graphic representation showing the effect of HYB 165 with or without docetaxel on the growth of ZR-75-1 human breast cancer cells.

FIG. 11 is a graphic representation showing the effect of HYB 508 with or without docetaxel on the growth of ZR-75-1 human breast cancer cells.

FIG. 12 is a graphic representation showing the effect of HYB 165 with or without monoclonal antibody MAb C225 on the growth of ZR-75-1 human breast cancer cells.

FIG. 13 is a graphic representation showing the effect of HYB 165 and HYB 295 on the growth of ZR-75-1 human breast cancer cells.

FIG. 14 is a graphic representation showing the effect of HYB 165 and HYB 508 on the growth of ZR-75-1 human breast cancer cells.

FIG. 15 is a graphic representation showing the effect of HYB 165 and HYB 295 on the growth of GEO colon cancer cells.

FIG. 16A is a graphic representation of data showing that the hybrid MBO antisense RIα inhibits tumor growth after i.p. administration.

FIG. 16B is a graphic representation of data showing that the hybrid MBO antisense RIα inhibits tumor growth after oral administration.

FIG. 17A is a graphic representation of data showing that oral hybrid MBO antisense RIα cooperatively inhibits tumor growth with taxol.

FIG. 17B is a graphic representation of data showing that oral hybrid MBO antisense RIα cooperatively increases survival in combination with taxol.

FIG. 18 is a tabular representation of histochemical analysis of GEO tumors following treatment with taxol and/or different oral MBOs.

### DESCRIPTION OF THE INVENTION

The patent and scientific literature referred to herein establishes the knowledge that is available to those with skill in the art. The issued U.S. patents, applications, published foreign applications, and references cited herein are hereby incorporated by reference.

In some embodiments the present invention is directed to methods and therapeutic compositions for inhibiting cancer cell proliferation or treating cancer. The methods and pharmaceutical compositions of some embodiments of the invention include the use of modified oligonucleotides that down-regulate the expression of the PKA RIα gene in combination with a second therapeutic agent such as a topoisomerase I inhibitor.

The modified oligonucleotides useful in the methods and compositions of the invention are synthetic oligonucleotides that are hybrid, inverted hybrid, or inverted chimeric. As used herein “hybrid oligonucleotide” means an oligonucleotide comprising at least one region of one or more deoxynucleotides and at least one region of one or more ribonucleotides. As used herein, the term “inverted hybrid oligonucleotide” means an oligonucleotide comprising a region of one or more ribonucleotides flanked by 3′ and 5′ deoxyribonucleotide regions of one or more deoxynucleotides. As used herein, the term “chimeric oligonucleotide” means an oligonucleotide comprising more than one type of internucleotide linkages. In one embodiment a chimeric oligonucleotide has at least one region with ionic internucleotide linkages and at least one region with nonionic internucleotide linkages. In one nonlimiting example, a chimeric oligonucleotide has a segment of nonionic internucleotide linkages as well as having phosphorothioate internucleotide linkages at ionic positions. As used herein, the term “inverted chimeric oligonucleotide” means an oligonucleotide comprising a region of one or more nonionic internucleotide linkages flanked by regions of one or more ionic internucleotide linkages.

Such synthetic hybrid, inverted hybrid, and inverted chimeric oligonucleotides utilized in various embodiments of the invention have a nucleotide sequence complementary to a genomic region, or an RNA molecule transcribed therefrom, encoding the RIα subunit of PKA. The sequence of the PKA gene is known. An oligonucleotide utilized according to various embodiments of the invention can have any nucleotide sequence complementary to any region of the gene. In certain embodiments the oligonucleotides are about 18 nucleotides long. Three non-limiting examples of an 18 mer utilized according to various embodiments of the invention has the sequence set forth below in TABLE 1 as SEQ ID NOS:1, 4, and 6.

In some embodiments, the oligonucleotide is a hybrid oligonucleotide comprising a region of at least two deoxynucleotides, flanked by 5′ and 3′ ribonucleotide regions, each having at least four ribonucleotides. A hybrid oligonucleotide having the sequence set forth in the Sequence Listing as SEQ ID NO:4 is one nonlimiting embodiment. In some embodiments, each of the 3′ and 5′ flanking ribonucleotide regions of an oligonucleotide of the invention comprises at least four contiguous, 2′-O-substituted ribonucleotides. In some embodiments, each of the 3′ and 5′ flanking ribonucleotide regions of an oligonucleotide of the invention comprises at least one 2′-O-alkyl substituted ribonucleotide. In one useful embodiment, the 2′-O-alkyl-substituted nucleotide is a 2′-O-methyl ribonucleotide. In other useful embodiments, the 3′ and 5′ flanking ribonucleotide regions of an oligonucleotide of the invention comprise at least four 2′-O-methyl ribonucleotides. In some embodiments, the ribonucleotides and deoxyribonucleotides of the hybrid oligonucleotide are linked by phosphorothioate internucleotide linkages. In some embodiments, this phosphorothioate region or regions have from about four to about 18 nucleosides joined to each other by 5′ to 3′ phosphorothioate linkages. In some embodiments, this phosphorothioate region or regions have from about 5 to about 18 such phosphorothioate-linked nucleosides. The phosphorothioate linkages may be mixed Rₚ and Sₚ enantiomers, or they may be stereoregular or substantially stereoregular in either Rₚ or Sₚ form (see Iyer et al. (1995) Tetrahedron Asymmetry 6:1051-1054).

In other embodiments, the oligonucleotide is an inverted hybrid oligonucleotide comprising a region of at least four ribonucleotides flanked by 3′ and 5′ deoxyribonucleotide regions of at least two deoxynucleotides. The structure of an inverted hybrid oligonucleotide is “inverted” relative to traditional hybrid oligonucleotides. In some embodiments, a 2′-O-substituted RNA region has from about four to about ten 2′-O-substituted nucleosides joined to each other by 5′ to 3′ internucleoside linkages, and in some embodiments it has from about four to about six such 2′-O-substituted nucleosides. In some embodiments, the oligonucleotides of the invention have a ribonucleotide region that comprises at least five contiguous ribonucleotides. In one embodiment, the overall size of the inverted hybrid oligonucleotide is 18. In some embodiments, the 2′-O-substituted ribonucleosides are linked to each other through a 5′ to 3′ phosphorothioate, phosphorodithioate, phosphotriester, or phosphodiester linkages. The phosphorothioate 3′ or 5′ flanking region (or regions) has from about four to about 18 nucleosides joined to each other by 5′ to 3′ phosphorothioate linkages, and in some embodiments it has from about 5 to about 18 such phosphorothioate-linked nucleosides. In some embodiments, the phosphorothioate regions have at least 5 phosphorothioate-linked nucleosides. One embodiment is an oligonucleotide having substantially the nucleotide sequence set forth in the Sequence Listing as SEQ ID NO:6. In some embodiments, the ribonucleotide region comprises 2′-O-substituted ribonucleotides, such as 2′-O-alkyl substituted ribonucleotides. One embodiment is an inverted hybrid oligonucleotide whose ribonucleotide region comprises at least one 2′-O-methyl ribonucleotide.

In some embodiments, all of the nucleotides in the inverted hybrid oligonucleotide are linked by phosphorothioate internucleotide linkages. In some embodiments, the deoxyribonucleotide flanking region or regions has from about four to about 18 nucleosides joined to each other by 5′ to 3′ phosphorothioate linkages, and in some embodiments it has from about 5 to about 18 such phosphorothioate-linked nucleosides. In some embodiments, the deoxyribonucleotide 3′ and 5′ flanking regions of the inverted hybrid oligonucleotides of the invention have about 5 phosphorothioate-linked nucleosides. The phosphorothioate linkages may be mixed Rₚ and Sₚ enantiomers, or they may be stereoregular or substantially stereoregular in either Rₚ or Sₚ form (see Iyer et al. (1995) Tetrahedron Asymmetry 6:1051-1054).

In other embodiments, the oligonucleotide is an inverted chimeric oligonucleotide comprising an oligonucleotide nonionic region of at least four nucleotides flanked by one or more, and in certain embodiments by two, oligonucleotide phosphorothioate regions. The structure of such an inverted chimeric oligonucleotide is “inverted” relative to traditional chimeric oligonucleotides. In one embodiment, an inverted chimeric oligonucleotide of the invention has substantially the nucleotide sequence set forth in the Sequence Listing as SEQ ID NO:1. In certain embodiments, the oligonucleotide nonionic region comprises about four to about 12 nucleotides joined to each other by 5′ to 3′ nonionic linkages. In some embodiments, the nonionic region contains alkylphosphonate and/or phosphoramidate and/or phosphotriester internucleoside linkages. In one embodiment, the oligonucleotide nonionic region comprises six nucleotides. In some embodiments, the oligonucleotide has a nonionic region having from about six to about eight methylphosphonate-linked nucleosides, flanked on either side by phosphorothioate regions, each having from about six to about ten phosphorothioate-linked nucleosides. In some embodiments, the flanking region or regions are phosphorothioate nucleotides. In certain embodiments, the flanking region or regions have from about four to about 24 nucleosides joined to each other by 5′ to 3′ phosphorothioate linkages, and in some embodiments from about six to about 16, such phosphorothioate-linked nucleosides. In some embodiments, the phosphorothioate regions have from about five to about 15 phosphorothioate-linked nucleosides. The phosphorothioate linkages may be mixed Rₚ and Sₚ enantiomers, or they may be stereoregular or substantially stereoregular in either Rₚ or Sₚ form (see Iyer et al. (1995) Tetrahedron Asymmetry 6:1051-1054).

Those skilled in the art will recognize that the elements of these oligonucleotide embodiments can be combined and the invention does contemplate such combination. For example, 2′-O-substituted ribonucleotide regions may well include from one to all nonionic internucleoside linkages. Alternatively, nonionic regions may have from one to all 2′-O-substituted ribonucleotides. Moreover, oligonucleotides utilized according to various embodiments of the invention may contain combinations of one or more 2′-O-substituted ribonucleotide region and one or more nonionic region, either or both being flanked by phosphorothioate regions. (See Nucleosides & Nucleotides 14:1031-1035 (1995) for relevant synthetic techniques.)

TABLE 1

| Oligo # | Sequence (5′ → 3′) | Type | SEQ ID NO: |
|---|---|---|---|
| 164 | GCG TGC CTC CTC ACT GGC | Antisense | 1 |
| 167 | GCG CGC CTC CTC GCT GGC | Mismatched Control | 2 |
| 188 | GCA TGC TTC CAC ACA GGC | Mismatched Control | 3 |
| | *** * * *** | | |
| 165 | GCG UGC CTC CTC ACU GGC | Hybrid | 4 |
| | *** * * *** | Mismatched Hybrid | |
| 168 | GCG CGC CTC CTC GCU GGC | (Control) | 5 |
| | *** ** | | |
| 166 | GCG TGC CUC CUC ACT GGC | Inverted Hybrid | 6 |
| | *** ** | Mismatched | |
| 169 | GCG CGC CUC CUC GCT GGC | Inverted Hybrid | 7 |
| | | (Control) | |
| | *** ** | Mismatched | |
| 189 | GCA TGC AUC CGC ACA GGC | Inverted Hybrid | 8 |
| | | (Control) | |
| | ... ... | | |
| 190 | GCG TGC CTC CTC ACT GGC | Inverted Chimeric | 1 |
| | ... ... | Mismatched | |
| 191 | GCG CGC CTC CTC GCT GGC | Inverted Chimeric | 2 |
| | | (Control) | |

X = mismatched bases
* ribonucleotide
. methylphosphonate nucleotide

Oligonucleotides having greater than 18 oligonucleotides are also contemplated by the invention. These oligonucleotides have up to 25 additional nucleotides extending from the 3′ terminus, or the 5′ terminus, or from both the 3′ and 5′ termini of, for example, the 18mer with SEQ ID NO:1, 4, or 6, without diminishing the ability of these oligonucleotides to down-regulate RIα gene expression. In some embodiments, the oligonucleotides are about 15 to about 30 nucleotides in length. In some embodiments they are about 15 to 25 nucleotides in length. In some embodiments the oligonucleotides are from about 13 to about 100 nucleotides in length. In certain embodiments the oligonucleotides are from about 15 to about 50 nucleotides in length, and in some embodiments the oligonucleotides are from about 15 to about 35 nucleotides in length. Alternatively, other oligonucleotides of the invention may have fewer nucleotides than, for example, oligonucleotides having SEQ ID NO:1, 4, or 6. Such shortened oligonucleotides maintain at least the antisense activity of the parent oligonucleotide to down-regulate the expression of the RIα gene, or have greater activity.

The oligonucleotides of the invention can be prepared by art recognized methods. Oligonucleotides with phosphorothioate linkages can be prepared manually or by an automated synthesizer and then processed using methods well known in the field such as phosphoramidite (reviewed in Agrawal et al. (1992) Trends Biotechnol. 10:152-158, see, e.g., Agrawal et al. (1988) Proc. Natl. Acad. Sci.(USA) 85:7079-7083) or H-phosphonate (see, e.g., Froehler (1986) Tetrahedron Lett. 27:5575-5578) chemistry. The synthetic methods described in Bergot et al. (J. Chromatog. (1992) 559:35-42) can also be used. Examples of other chemical groups include alkylphosphonates, phosphorodithioates, alkylphosphonothioates, phosphoramidates, 2′-O-methyls, carbamates, acetamidate, carboxymethyl esters, carbonates, and phosphate triesters. Oligonucleotides with these linkages can be prepared according to known methods (see, e.g., Goodchild (1990) Bioconjugate Chem. 2:165-187; Agrawal et al. (1988) Proc. Natl. Acad. Sci. (USA) 85:7079-7083; Uhlmann et al. (1990) Chem. Rev. 90:534-583; and Agrawal et al. (1992) Trends Biotechnol. 10:152-158).

Useful hybrid, inverted hybrid, and inverted chimeric oligonucleotides utilized according to various embodiments the invention may have other modifications which do not substantially affect their ability to specifically down-regulate RIα gene expression. These modifications include those which are internal or are at the end(s) of the oligonucleotide molecule and include additions to the molecule at the internucleoside phosphate linkages, such as cholesteryl or diamine compounds with varying numbers of carbon residues between the two amino groups, and terminal ribose, deoxyribose and phosphate modifications which cleave or crosslink to the opposite chains or to associated enzymes or other proteins which bind to the RIα nucleic acid. Examples of such oligonucleotides include those with a modified base and/or sugar such as arabinose instead of ribose, or a 3′,5′-substituted oligonucleotide having a sugar which, at one or both its 3′ and 5′ positions is attached to a chemical group other than a hydroxyl or phosphate group (at its 3′ or 5′ position). Other modified oligonucleotides are capped with a nuclease resistance-conferring bulky substituent at their 3′ and/or 5′ end(s), or have a substitution in one or both nonbridging oxygens per nucleotide. Such modifications can be at some or all of the internucleoside linkages, as well as at either or both ends of the oligonucleotide and/or in the interior of the molecule (reviewed in Agrawal et al. (1992) Trends Biotechnol. 10: 152-158).

In some embodiments the invention also provides therapeutic compositions suitable for treating undesirable, uncontrolled cell proliferation or cancer comprising at least one oligonucleotide in accordance with various embodiments of the invention, capable of specifically down-regulating expression of the RIα gene, and a pharmaceutically acceptable carrier or diluent. In some embodiments the oligonucleotide used in the therapeutic composition of the invention is complementary to at least a portion of the RIα genomic region, gene, or RNA transcript thereof. In some embodiments the invention provides a composition of matter that inhibits the expression of a protein kinase A subunit RIα with reduced side effects, the composition comprising an oligonucleotide (such as a hybrid, inverted hybrid, or inverted chimeric oligonucleotide) according to various embodiments of the invention.

As used herein, a “pharmaceutically or physiologically acceptable carrier” includes any and all solvents (including but not limited to lactose), dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of some embodiments of the invention is contemplated. Supplementary active ingredients can also be incorporated into the compositions.

Several useful therapeutic compositions of the invention suitable for inhibiting cell proliferation in vitro or in vivo or for treating cancer in humans in accordance with some of the methods of the invention comprise about 25 mg to 75 mg of a lyophilized oligonucleotide(s) having SEQ ID NOS:1, 4, and/or 6 and 20 mg to 75 mg lactose, USP, which is reconstituted with sterile normal saline to the therapeutically effective dosages described herein.

In some embodiments the invention also provides pharmaceutical compositions comprising a modified oligonucleotide according to various embodiments of the invention in combination with an antibody that binds to epidermal growth factor receptor (EGFR) or a cytotoxic agent. Useful cytotoxic agents include, without limitation, taxanes, platinum-derived agents, topoisomerase II-selective drugs, and topoisomerase I inhibitors. These classes of cytotoxic drugs can include prodrugs as well, including but not limited to, esters or amides of anti-cancer drugs, such as topoisomerase I inhibitors. Nonlimiting examples of prodrugs of such topoisomerase I inhibitors include Camptosar® analogs.

In additional embodiments the invention also provides methods for treating humans suffering from disorders or diseases wherein the RIα gene is incorrectly or over-expressed. Such a disorder or disease that could be treated using this method includes tumor-forming cancers such as, but not limited to, human colon carcinoma, breast carcinoma, gastric carcinoma, and neuroblastoma. In some methods of the invention, a therapeutically effective amount of a composition of some embodiments of the invention is administered to the human. Such methods of treatment, may be administered in conjunction with other therapeutic agents. Some embodiments of the invention also provide a method of treating cancer in an afflicted subject with reduced side effects. Such methods comprise administering a therapeutic composition of some embodiments of the invention to the subject in which the protein kinase A subunit RIα gene is being over-expressed.

In certain embodiments, the methods of treatment according to the invention comprise a) administering a first agent comprising a synthetic, modified oligonucleotide complementary to, and capable of down-regulating the expression of, nucleic acid encoding protein kinase A subunit RIα according to the invention; and b) administering a second agent comprising an antibody that binds to epidermal growth factor receptor (EGFR) or a cytotoxic agent selected from the group consisting of taxanes, platinum-derived agents, topoisomerase II-selective drugs, and topoisomerase I inhibitors. These classes of cytotoxic drugs can include prodrugs as well, including but not limited to, esters or amides of anti-cancer drugs, such as topoisomerase I inhibitors. Nonlimiting examples of prodrugs of such topoisomerase I inhibitors include Camptosar® analogs. In some embodiments according to this aspect of the invention, the two agents are administered simultaneously. In certain embodiments, the second agent is administered prior to administration of the first agent.

In some embodiments, the second agent is a taxane, including but not limited to paclitaxel and docetaxel. Paclitaxel may be administered in doses of up to 300 mg/m²/dose by intravenous infusion (1 hour to 24 hour duration), given at a frequency of every 21 days or less. Docetaxel can be administered in doses of up to 300 mg/m²/dose by intravenous infusion (1 hour to 24 hour duration), given at a frequency of every 21 days or less.

In certain other embodiments, the second agent is an antibody that binds to epidermal growth factor receptor. The antibody may be a monoclonal antibody, such as a humanized monoclonal antibody. In certain embodiments, the monoclonal antibody is C225 (N. I. Goldstein et al., Clin. Cancer Res., 1(11):1311-8 (1995). C225 may be administered in doses of up to 500 mg/m²/dose by intravenous infusion (10 minutes to 24 hour duration), given at a frequency of every 28 days or less.

In some embodiments, the second agent is a cytotoxic drug, which is a prodrug. As used herein, a “prodrug” is a compound comprising an active compound covalently linked to another moiety by a cleavable linkage, wherein the pharmacological activity of the active compound is greater than the pharmacological activity of the prodrug, and wherein the active compound is produced in the body by cleavage of the cleavable linkage. An “active compound” is a molecule having a pharmacological activity. A “pharmacological activity” is an activity that is useful in the treatment of one or more disease or disease symptom. A “moiety” is a chemical group or structure. A “cleavable linkage” is a covalent bond that can be cleaved by an enzymatic activity in the body. The term “co-administration” is intended to include treatment regimens in which either the prodrug or the oligonucleotide is continued after the cessation of the other agent.

Nonlimiting examples of useful prodrugs include amides and esters of active compounds. Such active compounds include, without limitation, anticancer chemotherapeutics, anti-inflammatory agents, antiinfectious agents, antiviral agents and cardiovascular drugs. Numerous prodrugs are well known in the art (see, e.g., Singh et al., J. Sci. Ind. Res. 55: 497-510 (1996)). A non-limiting example of such an active compound is SN-38, which is a topoisomerase I inhibitor. Specific non-limiting examples of prodrugs include Camptosar® ((7-ethyl-10-(-4-piperidinol)-1-piperidnocarbonyloxy-camptothecin; CPT-11) (also known as irinotecan) and foscarnate. The moiety that is cleaved from the prodrug may be selected from esters and alpha-acyloxyalkyl esters (for carboxy functionalities); amides, esters, carbonate esters, phosphate esters, ethers and alpha-acyloxyalkyl ethers (for hydroxy functionalities); thioesters, alpha-acyloxyalkyl thioesters and disulfides (for sulfhydryl functionalities); ketals, imines, enol esters, oxazoladines, and thiazolidines (for carbonyl functionalities); amides, carbamates, imines enamines N-Mannich bases, and N-acyloxyalkoxycarbonyl derivatives (for amino functionalities); N-acyloxyalkyl derivatives (for quaternary amino functionalities); N-sulphonyl imidates (for ester or sulfonamido functionalities); N-Mannich bases (for NH-acidic functionalities); and N-acyloxyalkyl derivatives (for heterocyclic amino functionalities).

Some embodiments of the invention provide methods for administering prodrugs in a manner that maximizes their efficacy, and thus allows lower, less toxic dosages to be used. The methods according to some embodiments of the invention act through a variety of mechanisms that modulate the ability of the body to process the prodrug to the active compound and its ability to clear either the prodrug or the active compound, and are thus applicable to a broad range of prodrugs.

Additional embodiments of the invention provide methods for statistically significantly potentiating the activity of a prodrug without producing significant side effects. In this method, an oligonucleotide is co-administered with the prodrug, wherein the prodrug is present in an amount that would not be therapeutically effective in the absence of the oligonucleotide. Methods according to this aspect of the invention are useful where toxicity of the prodrug or active compound is dose-limiting. Thus these methods can increase the therapeutic index for the prodrug.

While various embodiments of the invention relate to the use of an oligonucleotide, such as an antisense oligonucleotide complementary to PKA RIα, it also has been discovered that more generally the coadministration of a polyanion with a prodrug will statistically significantly potentiate the activity of the prodrug without producing significant side effects. Other polyanions that could be used include, but are not limited to, polysulfates and additional oligonucleotides. Examples of polysulfates include heparin, dextran sulfates, suramin sulfates, cyclodextrin sulfates and oligonucleotide phosphorothioates or phosphorodithioates. Examples of oligonucleotides that can also be used as polyanions in some methods also include double stranded oligonucleotides, including hairpin oligonucleotides, as well as cyclic oligonucleotides. Oligonucleotides in non-antisense embodiments can have the same ranges in length as antisense oligonucleotides, but they can also be from about 5 to about 15 nucleotides in length. The nucleic acid sequence to which the modified oligonucleotide sequence is complementary will depend upon the biological effect that is sought to be modified. In certain embodiments the oligonucleotide is complementary to a gene selected from mdm-2, PKA, PKC, raf-kinase, bcl-2, H-ras, c-myc, DNA methyltransferase, histone deacetylase and VEGF. By way of nonlimiting example, when an antisense oligonucleotide complementary to PKA RIα is used in combination with a prodrug such as Camptosar®, the oligonucleotide will exert both sequence specific as well as non-sequence specific potentiation.

The term “without producing significant side effects” means that any signs or symptoms of toxicity that are observed in the presence of the polyanion (such as an antisense oligonucleotide) are not greater than those observed in the absence of the polyanion to an extent that would preclude the combination of the prodrug and the polyanion from obtaining regulatory approval.

It also has been surprisingly discovered that administration of the polyanion prior to the administration of the prodrug results in even greater potentiation of the prodrug than when the polyanion is administered at the same time as, or after, administration of the prodrug.

Without wishing to be bound by theory, the potentiation of the prodrug is believed to involve one or more of the following mechanisms: modulation of the retention time of the prodrug in the liver and other tissues, including tumor tissue; competition with cleavage enzymes or other hepatic enzymes, e.g., carboxylesterases, amidases, or other esterases; competition with transport factors from the liver, e.g., cMOAT for CPT-11; competition for binding of serum proteins; competition with binding of endothelial cell walls; competition for covalent modification, e.g., glucouronidation; slowing hydrolysis of the prodrug so that active metabolite is continuously released into the blood circulation; and stabilization of the active form of the drug, e.g., lactone formation for CPT-11.

Any of these mechanisms may benefit by saturation of the system with the polyanion prior to administration of the prodrug.

In some embodiments according to the invention, the first agent is a synthetic modified oligonucleotide having a nucleotide sequence consisting essentially of the nucleotide sequence set forth in SEQ ID NO:4. The oligonucleotide may be administered at a dose of up to 540 mg/m²/dose by intravenous infusion (2 hours to 21 days in duration or up to 1,050 mg/m²/day by oral or rectal administration.

As used herein, the term “therapeutically effective amount” means the total amount of each active component of the pharmaceutical formulation or method that is sufficient to show a meaningful subject or patient benefit, e.g., a reduction in tumor growth or in the expression of proteins which cause or characterize the cancer. When applied to an individual active ingredient, administered alone, the term refers to that ingredient alone. When applied to a combination, the term refers to combined amounts of the active ingredients that result in the therapeutic effect, whether administered in combination, serially or simultaneously.

A “therapeutically effective manner” refers to a route, duration, and frequency of administration of the pharmaceutical formulation which ultimately results in meaningful patient benefit, as described above. In some embodiments of the invention, the pharmaceutical formulation is administered via injection, sublingually, rectally, intradermally, orally, or enterally in bolus, continuous, intermittent, or continuous followed by intermittent regimens.

The therapeutically effective amount of synthetic oligonucleotide in the pharmaceutical composition according to some embodiments of the present invention will depend upon the nature and severity of the condition being treated, and on the nature of prior treatments which the patient has undergone. Ultimately, the attending physician will decide the amount of synthetic oligonucleotide with which to treat each individual patient. Initially, the attending physician will administer low doses of the synthetic oligonucleotide and observe the patient's response. Larger doses of synthetic oligonucleotide may be administered until the optimal therapeutic effect is obtained for the patient, and at that point the dosage is not increased further. It is contemplated that the dosages of the pharmaceutical compositions administered in various methods of the present invention should contain about 0.1 mg/kg to 5.0 mg/kg body weight per day and in some embodiments 0.1 mg/kg to 2.0 mg/kg body weight per day. When administered systemically, the therapeutic composition may be administered at a sufficient dosage to attain a blood level of oligonucleotide from about 0.01 μM to about 10 μM. In some embodiments the concentration of oligonucleotide at the site of aberrant gene expression should be from about 0.01 μM to about 10 μM, and in some embodiments it is from about 0.05 μM to about 5 μM. However, for localized administration, much lower concentrations than this may be effective, and much higher concentrations may be tolerated. It may be desirable to administer simultaneously or sequentially a therapeutically effective amount of one or more of the therapeutic compositions according to various embodiments of the invention to an individual as a single treatment episode.

Administration of pharmaceutical compositions in accordance with some embodiments of the invention or to practice embodiments of the methods of the present invention can be carried out in a variety of conventional ways, such as by oral ingestion, enteral, rectal, or transdermal administration, inhalation, sublingual administration, or cutaneous, subcutaneous, intramuscular, intraocular, intraperitoneal, or intravenous injection, or any other route of administration known in the art for administrating therapeutic agents.

When the composition is to be administered orally, sublingually, or by any non-injectable route, the therapeutic formulation may include a physiologically acceptable carrier, such as an inert diluent or an assimilable edible carrier with which the composition is administered. Suitable formulations that include pharmaceutically acceptable excipients for introducing compounds to the bloodstream by other than injection routes can be found in Remington's Pharmaceutical Sciences (18th ed.) (Genarro, ed. (1990) Mack Publishing Co., Easton, Pa.). The oligonucleotide and other ingredients may be enclosed in a hard or soft shell gelatin capsule, compressed into tablets, or incorporated directly into the individual's diet. The therapeutic compositions may be incorporated with excipients and used in the form of ingestible tablets, buccal tablets, troches, capsules, elixirs, suspensions, syrups, wafers, and the like. When the therapeutic composition is administered orally, it may be mixed with other food forms and pharmaceutically acceptable flavor enhancers. When the therapeutic composition is administered enterally, they may be introduced in a solid, semi-solid, suspension, or emulsion form and may be compounded with any number of well-known, pharmaceutically acceptable additives. Sustained release oral delivery systems and/or enteric coatings for orally administered dosage forms are also contemplated such as those described in U.S. Pat. Nos. 4,704,295, 4,556,552, 4,309,404, and 4,309,406.

When a therapeutically effective amount of composition according to some embodiments of the invention is administered by injection, the synthetic oligonucleotide may be in the form of a pyrogen-free, parenterally-acceptable, aqueous solution. The preparation of such parenterally-acceptable solutions, having due regard to pH, isotonicity, stability, and the like, is within the skill in the art. A useful pharmaceutical composition for injection should contain, in addition to the synthetic oligonucleotide, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of some embodiments of the present invention may also contain stabilizers, preservatives, buffers, antioxidants, or other additives known to those of skill in the art.

The pharmaceutical forms suitable for injectable use include sterile aqueous solutions or dispersions and sterile powders for the extemporaneous preparation of sterile injectable solutions or dispersions. In all cases the form should be sterile. It should be stable under the conditions of manufacture and storage and may be preserved against the contaminating action of microorganisms, such as bacteria and fungi. The carrier can be a solvent or dispersion medium. The prevention of the action of microorganisms can be brought about by various antibacterial and antifungal agents. Prolonged absorption of the injectable therapeutic agents can be brought about by the use of the compositions of agents delaying absorption. Sterile injectable solutions are prepared by incorporating the oligonucleotide in the required amount in the appropriate solvent, followed by filtered sterilization.

The pharmaceutical formulation can be administered in bolus, continuous, or intermittent dosages, or in a combination of continuous and intermittent dosages, as determined by the physician and the degree and/or stage of illness of the patient. The duration of therapy using the pharmaceutical composition of some embodiments of the present invention will vary, depending on the unique characteristics of the oligonucleotide and the therapeutic effect to be achieved, the limitations inherent in the art of preparing such a therapeutic formulation for the treatment of humans, the severity of the disease being treated and the condition and potential idiosyncratic response of each individual patient. Ultimately the attending physician will decide on the appropriate duration of intravenous therapy using the pharmaceutical composition of some embodiments of the present invention.

Some embodiments of compositions of the invention are useful for inhibiting or reducing the proliferation of cancer or tumor cells in vivo or in vitro. A synthetic oligonucleotide according to some embodiments of the invention is administered to the cells in an amount sufficient to enable the binding of the oligonucleotide to a complementary genomic region or RNA molecule transcribed therefrom encoding the RIα subunit. In this way, expression of PKA is decreased, thus inhibiting or reducing cell proliferation.

Compositions according to some embodiments of the invention are also useful for treating cancer or uncontrolled cell proliferation in humans. In this method, a therapeutic formulation including an antisense oligonucleotide according to various embodiments of the invention is provided in a physiologically acceptable carrier. The individual is then treated with the therapeutic formulation in an amount sufficient to enable the binding of the oligonucleotide to the PKA RIα genomic region or RNA molecule transcribed therefrom in the infected cells. In this way, the binding of the oligonucleotide inhibits or down-regulates RIα expression and hence the activity of PKA.

In practicing the method of treatment or use according to various embodiments of the present invention, a therapeutically effective amount of at least one or more therapeutic compositions of various embodiments of the invention is administered to a subject afflicted with a cancer. An anticancer response showing a decrease in tumor growth or size or a decrease in RIα expression is considered to be a positive indication of the ability of the method and pharmaceutical formulation to inhibit or reduce cell growth and thus, to treat cancer in humans.

At least one therapeutic composition of the invention may be administered in accordance with at least one of the methods of the invention either alone or in combination with other known therapies for cancer such as cisplatin, carboplatin, paclitaxel, tamoxifen, taxol, interferon α, doxorubicin, and CPT-11. When co-administered with one or more other therapies, the compositions of various embodiments of the invention may be administered either simultaneously with the other treatment(s), or sequentially. If administered sequentially, the attending physician will decide on the appropriate sequence of administering compositions according to various embodiments of the invention in combination with the other therapy.

The following examples illustrate various modes of making and practicing the present invention, but are not meant to limit the scope of the invention since alternative methods may be utilized to obtain similar results.

### EXAMPLE 1

### Synthesis, Deprotection, and Purification of Oligonucleotides

Oligonucleotides were synthesized using standard phosphoramidite chemistry (Beaucage (1993) Meth. Mol. Biol. 20:33-61) on an automated DNA synthesizer (Model 8700, Biosearch, Bedford, Mass.) using a beta-cyanoethyl phosphoramidate approach.

Oligonucleotide phosphorothioates were synthesized using an automated DNA synthesizer (Model 8700, Biosearch, Bedford, Mass.) using a beta-cyanoethyl phosphoramidate approach on a 10 micromole scale. To generate the phosphorothioate linkages, the intermediate phosphite linkage obtained after each coupling was oxidized using 3H, 1,2-benzodithiole-3H-one-1,1-dioxide (see Beaucage, in Protocols for Oligonucleotides and Analogs: Synthesis and Properties, Agrawal (ed.) (1993) Humana Press, Totowa, N.J., pp. 33-62). Similar synthesis was carried out to generate phosphodiester linkages, except that a standard oxidation was carried out using standard iodine reagent. Synthesis of inverted chimeric oligonucleotide was carried out in the same manner, except that methylphosphonate linkages were assembled using nucleoside methylphosphonamidite (Glen Research, Sterling, Va.), followed by oxidation with 0.1 M iodine in tetrahydrofuran/2,6-lutidine/water (75:25:0.25) (see Agrawal & Goodchild (1987) Tet. Lett. 28:3539-3542). Hybrids and inverted hybrid oligonucleotides were synthesized similarly, except that the segment containing 2′-O-methylribonucleotides was assembled using 2′-O-methylribonucleoside phosphoramidite, followed by oxidation to a phosphorothioate or phosphodiester linkage as described above. Deprotection and purification of oligonucleotides was carried out according to standard procedures, (see Padmapriya et al. (1994) Antisense Res. & Dev. 4:185-199), except for oligonucleotides containing methylphosphonate-containing regions. For those oligonucleotides, the CPG-bound oligonucleotide was treated with concentrated ammonium hydroxide for 1 hour at room temperature, and the supernatant was removed and evaporated to obtain a pale yellow residue, which was then treated with a mixture of ethylenediamine/ethanol (1:1 v/v) for 6 hours at room temperature and dried again under reduced pressure.

### EXAMPLE 2

### In Vitro Complement Activation Studies

To determine the relative effect of inverted hybrid or inverted chimeric structure on oligonucleotide-mediated depletion of complement, the following experiments were performed. Venous blood was collected from healthy adult human volunteers. Serum was prepared for hemolytic complement assay by collecting blood into vacutainers (Becton Dickinson #6430 Franklin Lakes, N.J.) without commercial additives. Blood was allowed to clot at room temperature for 30 minutes, chilled on ice for 15 minutes, then centrifuged at 4° C. to separate serum. Harvested serum was kept on ice for same day assay or, alternatively, stored at −70° C. Buffer, or an oligonucleotide sample was then incubated with the serum. The oligonucleotides tested were 25 mer oligonucleotide phosphodiesters or phosphorothioates, 25 mer hybrid oligonucleotides, 25 mer inverted hybrid oligonucleotides, 25 mer chimeric oligonucleotides, and 25 mer inverted chimeric oligonucleotides. Representative hybrid oligonucleotides were composed of seven to 13 2′-O-methyl ribonucleotides flanked by two regions of six to nine deoxyribonucleotides each. Representative 25 mer inverted hybrid oligonucleotides were composed of 17 deoxyribonucleotides flanked by two regions of four ribonucleotides each. Representative 25 mer chimeric oligonucleotides were composed of six methylphosphonate deoxyribonucleotides and 19 phosphorothioate deoxyribonucleotides. Representative inverted chimeric oligonucleotides were composed of from 16 to 17 phosphorothioate deoxyribonucleotides flanked by regions of from two to seven methylphosphonate deoxyribonucleotides, or from six to eight methylphosphonate deoxyribonucleotides flanked by nine to ten phosphorothioate deoxyribonucleotides, or two phosphorothioate regions ranging from two to 12 oligonucleotides, flanked by three phosphorothioate regions ranging in size from two to six nucleotides in length. A standard CH50 assay (See Kabat and Mayer (eds), Expt'al. Immunochem., 2d Ed., Springfield, Ill., C C Thomas, p. 125) for complement-mediated lysis of sheep red blood cells (Colorado Serum Co.) sensitized with anti-sheep red blood cell antibody (hemolysin, Diamedix, Miami, Fla.) was performed, using duplicate determinations of at least five dilutions of each test serum, then hemoglobin release into cell-free supernates was measured spectrophotometrically at 541 nm.

### EXAMPLE 3

### In Vitro Mitogenicity Studies

To determine the relative effect of inverted hybrid or inverted chimeric structure on oligonucleotide-mediated mitogenicity, the following experiments were performed. Spleen was taken from a male CD1 mouse (4-5 weeks, 20-22 g; Charles River, Wilmington, Mass.). Single cell suspensions were prepared by gently mincing with frosted edges of glass slides. Cells were then cultured in RPMI complete media (RPMI media supplemented with 10% fetal bovine serum (FBS), 50 micromolar 2-mercaptoethanol (2-ME), 100 U/ml penicillin, 100 micrograms/ml streptomycin, 2 mM L-glutamine). To minimize oligonucleotide degradation, FBS was first heated for 30 minutes at 65° C. (phosphodiester-containing oligonucleotides) or 56° C. (all other oligonucleotides). Cells were plated in 96 well dishes at 100,000 cells per well (volume of 100 microliters/well). One type of each oligonucleotide described in Example 2 above in 10 microliters TE buffer (10 mM Tris-HCl, pH 7.5, 1 mM EDTA) was added to each well. After 44 hours of culturing at 37° C., one microcurie tritiated thymidine (Amersham, Arlington Heights, Ill.) was added in 20 microliters RPMI media for a 4 hour pulse labeling. The cells were then harvested in an automatic cell harvester (Skatron, Sterling, Va.) and the filters were assessed using a scintillation counter. In control experiments for mitogenicity, cells were treated identically, except that either media (negative control) or concanavalin A (positive control) was added to the cells in place of the oligonucleotides.

All of the inverted hybrid oligonucleotides proved to be less immunogenic than phosphorothioate oligonucleotides. Inverted hybrid oligonucleotides having phosphodiester linkages in the 2′-O-methyl region appeared to be slightly less immunogenic than those containing phosphorothioate linkages in that region. No significant difference in mitogenicity was observed when the 2′-O-methyl ribonucleotide region was pared down from 13 to 11 or to 9 nucleotides. Inverted chimeric oligonucleotides were also generally less mitogenic than phosphorothioate oligonucleotides. In addition, these oligonucleotides appeared to be less mitogenic than traditional chimeric oligonucleotides, at least in cases in which the traditional chimeric oligonucleotides had significant numbers of methylphosphonate linkages near the 3′ end. Increasing the number of methylphosphonate linkers in the middle of the oligonucleotide from 5 to 6 or 7 did not appear to have a significant effect on mitogenicity. These results indicate that incorporation of inverted hybrid or inverted chimeric structure into an oligonucleotide can reduce its mitogenicity.

### EXAMPLE 4

### In Vitro Studies

To determine the relative effect of inverted hybrid or inverted chimeric structure on oligonucleotide-induced mitogenicity, the following experiments were performed. Venous blood was collected from healthy adult human volunteers. Plasma for clotting time assay was prepared by collecting blood into siliconized vacutainers with sodium citrate (Becton Dickinson #367705), followed by two centrifugations at 4° C. to prepare platelet-poor plasma. Plasma aliquots were kept on ice, spiked with various test oligonucleotides described in Example 2 above, and either tested immediately or quickly frozen on dry ice for subsequent storage at −20° C. prior to coagulation assay. Activated partial thromboplastin time (aPTT) was performed in duplicate on an Electra 1000C (Medical Laboratory Automation, Mount Vernon, N.Y.) according to the manufacturer's recommended procedures, using Actin FSL (Baxter Dade, Miami, Fla.) and calcium to initiate clot formation, which was measured photometrically. Prolongation of aPTT was taken as an indication of clotting inhibition side effect produced by the oligonucleotide.

Traditional phosphorothioate oligonucleotides produced the greatest prolongation of aPTT, of all of the oligonucleotides tested. Traditional hybrid oligonucleotides produced somewhat reduced prolongation of aPTT. In comparison with traditional phosphorothioate or traditional hybrid oligonucleotides, all of the inverted hybrid oligonucleotides tested produced significantly reduced prolongation of aPTT. Inverted hybrid oligonucleotides having phosphodiester linkages in the 2′-O-substituted ribonucleotide region had the greatest reduction in this side effect, with one such oligonucleotide having a 2′-O-methyl RNA phosphodiester region of 13 nucleotides showing very little prolongation of aPTT, even at oligonucleotide concentrations as high as 100 micrograms/ml. Traditional chimeric oligonucleotides produce much less prolongation of aPTT than do traditional phosphorothioate oligonucleotides. Generally, inverted chimeric oligonucleotides retain this characteristic. At least one inverted chimeric oligonucleotide, having a methylphosphonate region of seven nucleotides flanked by phosphorothioate regions of nine nucleotides, gave better results in this assay than the traditional chimeric oligonucleotides at all but the highest oligonucleotide concentrations tested. These results indicate that inverted hybrid and inverted chimeric oligonucleotides may provide advantages in reducing the side effect of clotting inhibition when they are administered to modulate gene expression in vivo.

### EXAMPLE 5

### In Vivo Complement Activation Studies

Rhesus monkeys (4-9 kg body weight) are acclimatized to laboratory conditions for at least 7 days prior to the study. On the day of the study, each animal is lightly sedated with ketamine-HCl (10 mg/kg) and diazepam (0.5 mg/kg). Surgical level anesthesia is induced and maintained by continuous ketamine intravenous drip throughout the procedure. The oligonucleotides described in Example 2 above are dissolved in normal saline and infused intravenously via a cephalic vein catheter, using a programmable infusion pump at a delivery rate of 0.42 mg/minute. For each oligonucleotide, doses of 0, 0.5, 1, 2, 5 and 10 mg/kg are administered to two animals each over a 10 minute infusion period. Arterial blood samples are collected 10 minutes prior to oligonucleotide administration and 2, 5, 10, 20, 40 and 60 minutes after the start of the infusion, as well as 24 hours later. Serum is used for determining complement CH50, using the conventional complement-dependent lysis of sheep erythrocyte procedure (see Kabat and Mayer, 1961, supra). At the highest dose, phosphorothioate oligonucleotide causes a decrease in serum complement CH50 beginning within 5 minutes of the start of infusion. Inverted hybrid and chimeric oligonucleotides are expected to show a much reduced or undetectable decrease in serum complement CH50 under these conditions.

### EXAMPLE 6

### In Vivo Mitogenicity Studies

CD1 mice are injected intraperitoneally with a dose of 50 mg/kg body weight of oligonucleotide described in Example 2 above. Forty-eight hours later, the animals are euthanized and the spleens are removed and weighed. Animals treated with inverted hybrid or inverted hybrid oligonucleotides are expected to show no significant increase in spleen weight, while those treated with oligonucleotide phosphorothioates are expected to show modest increases in spleen weight.

### EXAMPLE 7

### In Vivo Clotting Studies

Rhesus monkeys are treated as in Example 5. From the whole blood samples taken, plasma for clotting assay is prepared, and the assay performed, as described in Example 4. It is expected that prolongation of aPTT will be substantially reduced for both inverted hybrid oligonucleotides and for inverted chimeric oligonucleotide, relative to traditional oligonucleotide phosphorothioates.

### EXAMPLE 8

### RNase H Activity Studies

To determine the ability of inverted hybrid oligonucleotides and inverted chimeric oligonucleotides to activate RNase H when bound to a complementary RNA molecule, the following experiments were performed. Each type of oligonucleotide described in Example 2 above was incubated together with a molar equivalent quantity of complimentary oligoribonucleotide (0.266 micromolar concentration of each), in a cuvette containing a final volume of 1 ml RNase H buffer (20 mM Tris-HCl, pH 7.5, 10 mM MgCl₂, 0.1 M KCl, 2% glycerol, 0.1 mM DTT). The samples were heated to 95° C., then cooled gradually to room temperature to allow annealing to form duplexes. Annealed duplexes were incubated for 10 minutes at 37° C., then 5 units RNase H was added and data collection commenced over a three hour period. Data was collected using a spectrophotometer (GBC 920, GBC Scientific Equipment, Victoria, Australia) at 259 nm. RNase H degradation was determined by hyperchromic shift.

As expected, phosphodiester oligonucleotides behaved as very good co-substrates for RNase H-mediated degradation of RNA, with a degradative half-life of 8.8 seconds. Phosphorothioate oligonucleotides produced an increased half-life of 22.4 seconds. Introduction of a 2′-O-methyl ribonucleotide segment at either end of the oligonucleotide further worsened RNase H activity (half-life=32.7 seconds). In contrast, introducing a 2′-O-methyl segment into the middle of the oligonucleotide (inverted hybrid structure) always resulted in improved RNase H-mediated degradation. When a region of 13 2′-O-methylribonucleoside phosphodiesters was flanked on both sides by phosphorothioate DNA, the best RNase H activity was observed, with a half-life of 7.9 seconds. Introduction of large blocks of methylphosphonate-linked nucleosides at the 3′ end of the oligonucleotide either had no effect or caused further deterioration of RNase H activity even when in a chimeric configuration. Introduction of methylphosphonate linked nucleosides at the 5′ end, however, improved RNase H activity, such as when in a chimeric configuration with a single methylphosphonate linker at the 3′ end (best half-life=8.1 seconds). All inverted chimeric oligonucleotides with methylphosphonate core regions flanked by phosphorothioate regions gave good RNase results, with a half-life range of 9.3 to 14.4 seconds. These results indicate that the introduction of inverted hybrid or inverted chimeric structure into phosphorothioate-containing oligonucleotides can restore some or all of the ability of the oligonucleotide to act as a co-substrate for RNase H, a useful attribute for an effective antisense agent.

### EXAMPLE 9

### Melting Temperature Studies

To determine the effect of inverted hybrid or inverted chimeric structure on stability of the duplex formed between an antisense oligonucleotide and a target molecule, the following experiments were performed. Thermal melting (Tm) data were collected using a spectrophotometer (GBC 920, GBC Scientific Equipment, Victoria, Australia), which has six 10 mm cuvettes mounted in a dual carousel. In the Tm experiments, the temperature was directed and controlled through a peltier effect temperature controller by a computer, using software provided by GBC, according to the manufacturer's directions. Tm data were analyzed by both the first derivative method and the midpoint method, as performed by the software. Tm experiments were performed in a buffer containing 10 mM PIPES, pH 7.0, 1 mM EDTA, 1 M NaCl. A refrigerated bath (VWR 1166, VWR, Boston, Mass.) was connected to the peltier-effect temperature controller to absorb the heat. Oligonucleotide strand concentration was determined using absorbance values at 260 nm, taking into account extinction coefficients.

### EXAMPLE 10

### Tumor Growth and Antisense Treatment

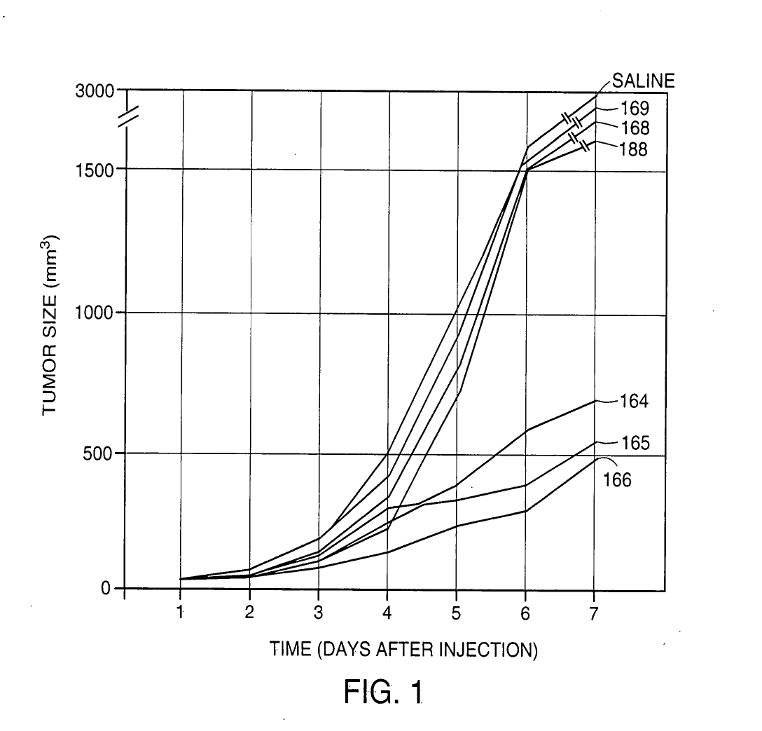

LS-174T human colon carcinoma cells (1×10⁶ cells) were inoculated subcutaneously (s.c.) into the left flank of athymic mice. A single dose of RIα, antisense hybrid (Oligo 165, SEQ ID NO:4), inverted hybrid (Oligo 166, SEQ ID NO:6), or antisense (Oligo 164, SEQ ID NO:1) oligonucleotides or control oligonucleotide (Oligo 169, (SEQ ID NO:7); Oligo 168 (SEQ ID NO:5); Oligo 188, (SEQ ID NO:3) as shown in Table 1 (1 mg per 0.1 ml saline per mouse), or saline (0.1 ml per mouse), was injected s.c. into the right flank of mice when tumor size reached 80 to 100 mg, about 1 week after cell inoculation. Tumor volumes were obtained from daily measurement of the longest and shortest diameters and calculation by the formula, 4/3πr³ where r=(length+width)/4. At each indicated time, two animals from the control and antisense-treated groups were killed, and tumors were removed and weighed. The results are shown in FIG. 1. These results show that the size of the tumor in the animal treated with the inverted hybrid oligonucleotide 166 having SEQ ID NO:6 was surprisingly smaller from three days after injection onward than the phosphorothioate oligonucleotide 164 having SEQ ID NO:1. That this effect was sequence-specific is also demonstrated in FIG. 1. Control oligonucleotide 168 (SEQ ID NO:5) has little ability to keep tumor size at a minimum relative to the hybrid and inverted hybrid oligonucleotides.

### EXAMPLE 11

### Photoaffmity Labeling and Immunoprecipitation of RI_{α} Subunits

The tumors are homogenized with a Teflon/glass homogenizer in ice-cold buffer 10 (Tris-HCl, pH 7.4, 20 mM; NaCl, 100 mM; NP-40, 1%; sodium deoxycholate, 0.5%; MgCl₂, 5 mM; pepstatin, 0.1 mM; antipain, 0.1 mM; chymostatin, 0.1 mM; leupeptin, 0.2 mM; aprotinin, 0.4 mg/ml; and soybean trypsin inhibitor, 0.5 mg/ml; filtered through a 0.45-μm pore size membrane), and centrifuged for 5 min in an Eppendorf microfuge at 4° C. The supernatants are used as tumor extracts.

The amount of PKA RIα subunits in tumors is determined by photoaffinity labeling with 8-N₃-[³²P] cAMP followed by immunoprecipitation with RIα antibodies as described by Tortora et al. (Proc. Natl. Acad. Sci. (USA) (1990) 87:705-708). The photoactivated incorporation of 8-N₃-[³²P] cAMP (60.0 Ci/m-mol), and the immunoprecipitation using the anti-RIα or anti-RII_{β} antiserum and protein A Sepharose and SDS-PAGE of solubilized antigen-antibody complex follows the method previously described (Tortora et al. (1990) Proc. Natl. Acad. Sci. (USA) 87:705-708; Ekanger et al. (1985) J. Biol. Chem. 260:3393-3401). It is expected that the amount of RIα in tumors treated with hybrid, inverted hybrid, and inverted chimeric oligonucleotides of the invention will be reduced compared with the amount in tumors treated with mismatch, straight phosphorothioate, or straight phosphodiester oligonucleotide controls, saline, or other controls.

### EXAMPLE 12

### cAMP-Dependent Protein Kinase Assays

Extracts (10 mg protein) of tumors from antisense-, control antisense-, or saline-treated animals are loaded onto DEAE cellulose columns (1×10 cm) and fractionated with a linear salt gradient (Rohlff et al. (1993) J. Biol. Chem. 268:5774-5782). PKA activity is determined in the absence or presence of 5 μM cAMP as described below (Rohlff et al. (1993) J. Biol. Chem. 268:5774-5782). cAMP-binding activity is measured by the method described previously and expressed as the specific binding (Tagliaferri et al. (1988) J. Biol. Chem. 263:409-416).

After two washes with Dulbecco's phosphate buffered saline, cell pellets (2×10⁶ cells) are lysed in 0.5 ml of 20 mM Tris (pH 7.5), 0.1 mM sodium EDTA, 1 mM dithiothreitol, 0.1 mM pepstatin, 0.1 mM antipain, 0.1 mM chymostatin, 0.2 mM leupeptin, 0.4 mg/ml aprotinin, and 0.5 mg/ml soybean trypsin inhibitor, using 100 strokes of a Dounce homogenizer. After centrifugation (Eppendorf 5412) for 5 min, the supernatants are adjusted to 0.7 mg protein/ml and assayed (Uhler et al. (1987) J. Biol. Chem. 262:15202-15207) immediately. Assays (40 μl total volume) are performed for 10 min at 300° C. and contained 200,μM ATP, 2.7×10⁶ cpm γ[³²P]ATP, 20 mM MgCl₂, 100 ,uM Kemptide (Sigma K-1127) (Kemp et al. (1977) J. Biol. Chem. 252:4888-4894), 40 mM Tris (pH 7.5), ±100,uM protein kinase inhibitor (Sigma P-3294) (Cheng et al. (1985) Biochem. J. 231:655-661), ±8,μM cAMP and 7 μg of cell extract. The phosphorylation of Kemptide is determined by spotting 20 μl of incubation mixture on phosphocellulose filters (Whatman, P81) and washing in phosphoric acid as described (Roskoski (1983) Methods Enzymol. 99:3-6). Radioactivity is measured by liquid scintillation using Econofluor-2 (NEN Research Products NEF-969). It is expected that PKA and cAMP binding activity will be reduced in extracts of tumors treated with the hybrid, inverted hybrid, and inverted chimeric oligonucleotides of the invention.

### EXAMPLE 13

### Effect of HYB 165 with Docetaxel and monoclonal Antibody MAb C225 on the Growth of ZR-75-1 Human Breast Cancer Cells

Materials: HYB 165, a 18-mer mixed backbone oligonucleotides (MBO) targeted against the N-terminal 8-13 codons of the human RIα regulatory subunit of PKA, synthesized by the procedure previously described was provided by Hybridon Inc., Cambridge, Mass. The antisense used had the following sequence: HYB 165, GCGUGCCTCCTCACUGGC, (SEQ ID NO:4) and contains 2-O-methyl-modified ribonucleotide bases (bold italics) at the 5′ and 3′ ends and unmodified oligodeoxynucleotide bases in the middle. Docetaxel was a kind gift from Rhone Poulenc Rorer, Origgio, Italy, and used after dilution in appropriate solvent as 10033 concentrated stock. The monoclonal antibody MAb C225 is a human-mouse chimeric IgG₁ that binds to the EGFR, competes with natural ligands for receptor binding and blocks the EGFR tyrosine kinase activation. Clinical grade MAb C225 was kindly provided by Dr. H. Waksal, ImClone Systems, New York, N.Y.

Cell lines: ZR-75-1 human breast cancer cells were purchased from American Type Culture Collection (Rockville, Md., USA). Cells were maintained in DMEM medium supplemented with 10% heat-inactivated FBS, 20 mM HEPES, pH 7.4, penicillin (100 UI/ml), streptomycin (100 μg/ml) and 4 mM glutamine (ICN, Irvine, UK) in a humidified atmosphere of 95% air and 5% CO₂ at 37° C.

Soft agar growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of docetaxel (day 0). HYB 165 and C225 were added together after 12 hrs (day 1) and on day 3. Twelve days after the last treatment, cells were stained with nitroblue tetrazolium (Sigma) and colonies larger than 0.05 mm were counted. Experiments were performed twice in triplicate.

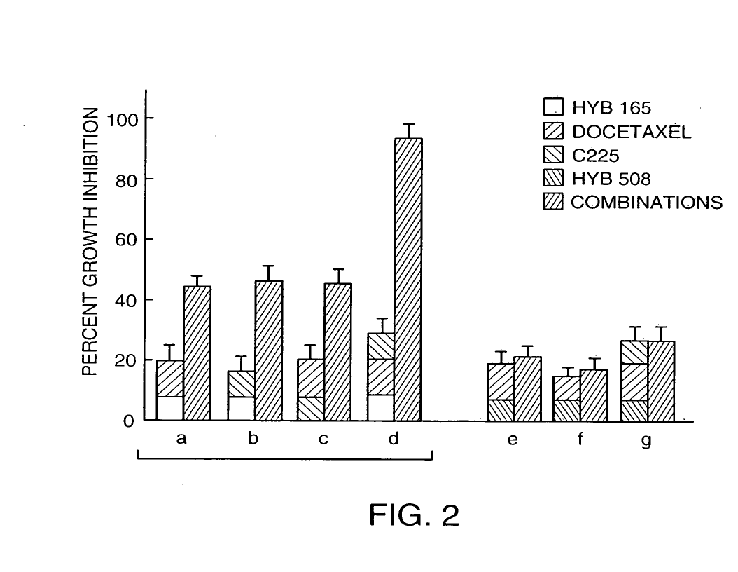

Results: HYB 165 0.1 μM, which alone causes about 8% inhibition and C225 0.25 μg/ml, which alone causes about 8% inhibition, were added to ZR-75-1 cells treated with docetaxel 0.01 nM, which alone causes less than 12% inhibition, determining an average 93% inhibition. See FIG. 2.

Conclusions: HYB 165, MAb C225 and docetaxel, at the low inhibitory doses of 0.1 μM, 0.25 μg/ml and 0.01 nM, respectively, cooperatively inhibit the growth of ZR-75-1 cells when used in combination. FIG. 2 shows the effect of the combination of HYB 165, the MAb C225 and Docetaxel on the soft agar growth of ZR-75-1 breast cancer cells. The doses of the different agents are: HYB 165, 0.1 and 0.5 μM; Docetaxel, 0.01 nM; MAb C225, 0.25 μg/ml. Data are expressed as percentage growth inhibition in reference to the growth of untreated control cells. The height of the bars on the left represents the sum of the individual agents effects and the expected percentage growth inhibition if drugs are additive when used in combination. The total height of the solid bar indicates the actual observed growth inhibition when drugs were used in combination. Therefore, the differences between the heights of the paired bars reflect the magnitude of synergism of growth inhibition. The data represent means and standard errors of triplicate determinations of two experiments.

### EXAMPLE 14

### Effect of HYB 508 with Docetaxel and Monoclonal Antibody MAb C225 on the Growth of ZR-75-1 Human Breast Cancer Cells

Materials: HYB 508, a 18-mer mixed backbone oligonucleotides (MBO) targeted against the N-terminal 8-13 codons of the human RIα regulatory subunit of PKA, synthesized by the procedure previously described was provided by Hybridon Inc., Cambridge, MA. The antisense used had the following sequence: HYB 508, GCAUGCTTCCACACAGGC, (SEQ ID NO:9) and contains 2-O-methyl-modified ribonucleotide bases (bold italics) at the 5′ and 3′ ends and unmodified oligodeoxynucleotide bases in the middle. HYB 508 is a control oligonucleotide of HYB 165, containing four mismatched nucleotides (underlined). Docetaxel was a kind gift from Rhone Poulenc Rorer, Origgio, Italy, and used after dilution in appropriate solvent as 100× concentrated stock. The monoclonal antibody MAb C225 is a human-mouse chimeric IgG, that binds to the EGFR, competes with natural ligands for receptor binding and blocks the EGFR tyrosine kinase activation. Clinical grade MAb C225 was kindly provided by Dr. H. Waksal, ImClone Systems, New York, N.Y.

Cell lines: ZR-75-1 human breast cancer cells were purchased from American Type Culture Collection (Rockville, Md., USA). Cells were maintained in DMEM medium supplemented with 10% heat-inactivated FBS, 20 mM HEPES, pH 7.4, penicillin (100 UI/ml), streptomycin (100 μg/ml) and 4 mM glutamine (ICN, Irvine, UK) in a humidified atmosphere of 95% air and 5% CO₂ at 37° C.

Soft agar growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of docetaxel (day 0). The HYB 508 and C225 were added together after 12 hrs (day 1) and on day 3. Twelve days after the last treatment, cells were stained with nitroblue tetrazolium (Sigma) and colonies larger than 0.05 mm were counted. Experiments were performed twice in triplicate.

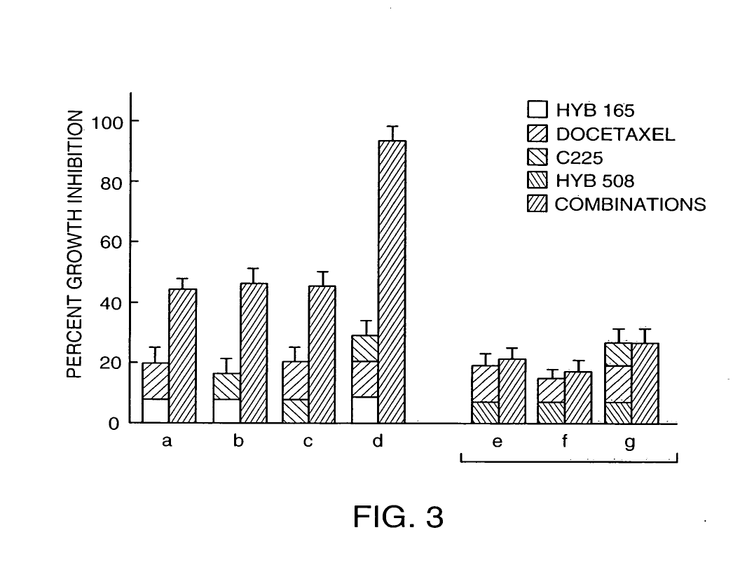

Results: HYB 508 0.5 μM, which alone causes about 6% inhibition and C225 0.25 μg/ml, which alone causes about 8% inhibition, were added to ZR-75-1 cells treated with docetaxel 0.01 nM, which alone causes about 12% inhibition, determining an average 26% inhibition. See FIG. 3.

Conclusions: HYB 508, MAb C225 and docetaxel, at the low inhibitory doses of 0.5 μM, 0.25 μg/ml and 0.01 nM, respectively, showed no cooperative antiproliferative effect on the growth of ZR-75-1 cells when used in combination. These results are shown in FIG. 3.

FIG. 3 shows the effect of the combination of Hyb 508, the MAb C225 and Docetaxel on the soft agar growth of ZR-75-1 breast cancer cells. The doses of the different agents are: HYB 508, 0.5 μM; Docetaxel, 0.01 nM; MAb C225, 0.25 μg/ml. Data are expressed as percentage growth inhibition in reference to the growth of untreated control cells. The height of the bars on the left represents the sum of the individual agents effects and the expected percentage growth inhibition if drugs are additive when used in combination. The total height of the solid bar indicates the actual observed growth inhibition when drugs were used in combination. Therefore, the differences between the heights of the paired bars reflect the magnitude of synergism of growth inhibition. The data represent means and standard errors of triplicate determinations of two experiments.

### EXAMPLE 15

### Effect HYB 165 with or without Paclitaxel on the Growth of Geo Human Colon Cancer Cells

Materials: HYB 165, a 18-mer mixed backbone oligonucleotides (MBO) targeted against the N-terminal 8-13 codons of the human RIα regulatory subunit of PKA, synthesized by the procedure previously described was provided by Hybridon Inc., Cambridge, Mass. The antisense used had the following sequence: HYB 165, GCGUGCCTCCTCACUGGC, (SEQ ID NO:4) and contains 2-O-methyl-modified ribonucleotide bases (bold italics) at the 5′ and 3′ ends and unmodified oligodeoxynucleotide bases in the middle. Paclitaxel was purchased from Sigma (St Louis, Mo.) and used after dilution in appropriate solvent as 100× concentrated stock.

Cell lines: GEO human colon cancer cells were purchased from American Type Culture Collection (Rockville, Md., USA). Cells were maintained in McCoy medium supplemented with 10% heat-inactivated FBS, 20 mM HEPES, pH 7.4, penicillin (100 UI/ml), streptomycin (100 μg/ml) and 4 mM glutamine (ICN, Irvine, UK) in a humidified atmosphere of 95% air and 5% CO. at 37° C.

Soft agar growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of paclitaxel (day 0). The HYB 165 was added after 12 hrs (day 1) and on day 2, 3 and 4. 12 days after the last treatment, cells were stained with nitroblue tetrazolium (Sigma) and colonies larger than 0.05 mm were counted. Experiments were performed twice in triplicate.

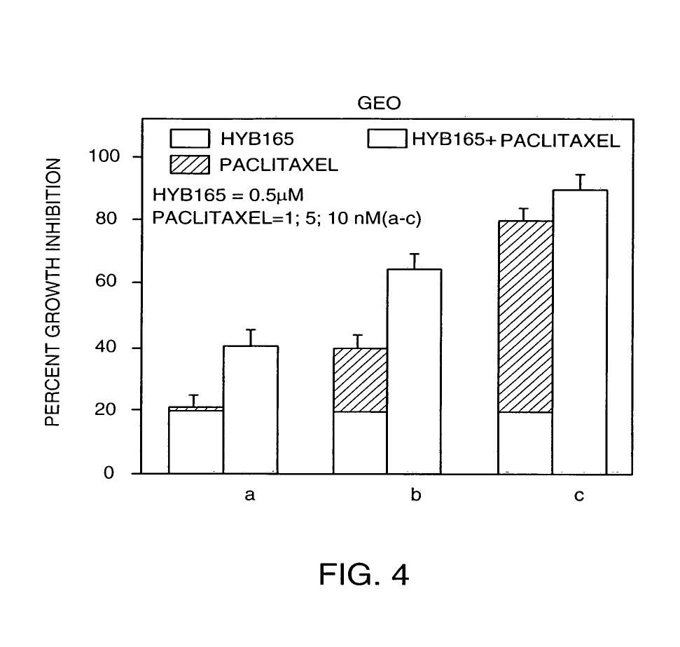

Results: A dose-dependent effect of paclitaxel at doses ranging between 0.1 and 10 nM was observed, determining up to about 60% growth inhibition. HYB 165 0.5 μM, which alone causes about 20% inhibition, was added to GEO cells treated with a) paclitaxel 1 nM, which alone causes less than 5% inhibition, determining an average 40% inhibition; b) paclitaxel 5 nM, which alone causes about 20% inhibition, determining an average 62% inhibition; c) paclitaxel 10 nM, which alone causes about 58% inhibition, determining an average 86% inhibition. See FIG. 4.

Conclusions: HYB 165 at the low inhibitory dose of 0.5 μM cooperatively inhibit the growth of GEO cells when used in a sequential combination with different doses of paclitaxel.

### EXAMPLE 16

### Effect of HYB 165 and its Control HYB 508 on the Growth of 1A9PTX22 Human Ovarian Cancer Cells

Materials: 18-mer mixed backbone oligonucleotides (MBO) targeted against the N-terminal 8-13 codons of the human RIα regulatory subunit of PKA, synthesized by the procedure previously described were provided by Hybridon Inc., Cambridge, Mass. The antisense used had the following sequences: HYB 165, GCGUGCCTCCTCACUGGC (SEQ ID NO:4); HYB 508, GCAUGCTTCCACACAGGC (SEQ ID NO:9). HYB 165 and HYB 508 are compounds containing 2-O-methyl-modified ribonucleotide bases (bold italics) at the 5′ and 3′ ends and unmodified oligodeoxynucleotide bases in the middle. HYB 508 is a control oligonucleotide containing four mismatched nucleotides as underlined.

Cell lines: The 1A9PTX22 cell line, a paclitaxel (PTX)-resistant clone of the human ovarian carcinoma cell line 1A9, was isolated by exposing 1A9 cells to 5 ng/ml PTX in the presence of 5 μg/ml verapamil, a P glycoprotein antagonist. 1A9PTX22 cells were kindly provided by Dr. Giannakakou, NCI Bethesda, Md., USA. Cells were maintained in RPMI medium supplemented with 10% heat-inactivated FBS, 20 mM HEPES, pH 7.4 penicillin (100 UI/ml), streptomycin (100 μg/ml) and 4 mM glutamine (ICN, Irvine, UK) 15 ng/ml PTX and 5 μg/ml verapamil in a humidified atmosphere of 95% air and 5% CO₂ at 37° C. 7 days before experiments were performed, PTX and verapamil were removed from culture medium.

Soft agar growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of HYB 165 or HYB 508 every 48 hours for three times. After 12 days the cells were stained with nitroblue tetrazolium (Sigma, St. Louis, Mo.) and colonies larger than 0.05 mm were counted. Experiments were performed twice in triplicate.

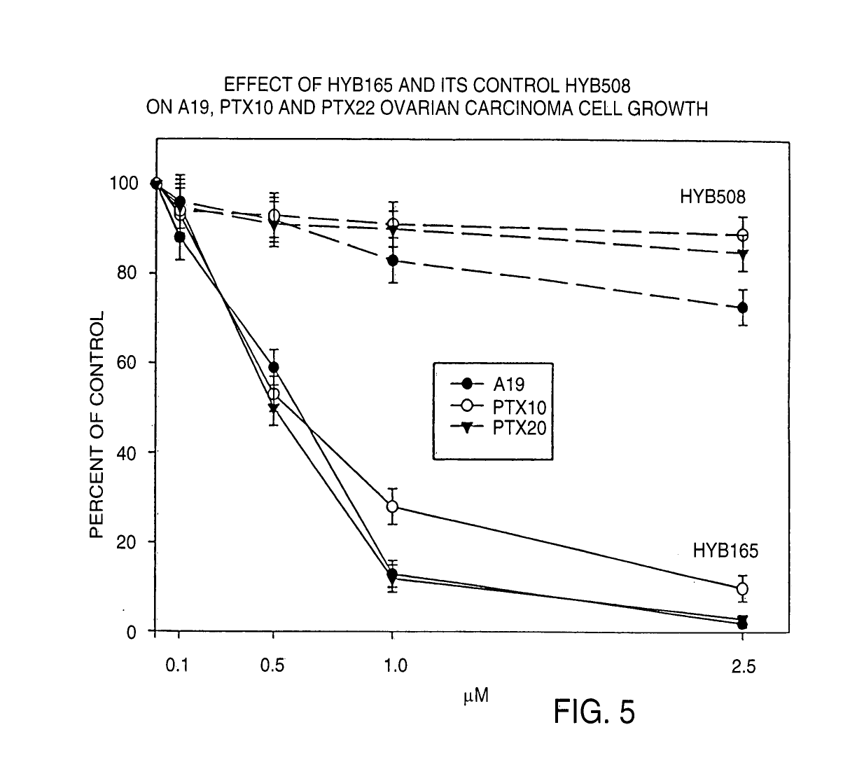

Results: Two different 18-mer MBOs complementary to the RIα subunit of PKA-I sequence, HYB 165 and its control oligomer HYB 508, differing only in 4 nucleotide bases, were tested to study their effect on soft agar growth of 1A9 human ovarian cancer cells. While HYB 165 determined a dose-dependent inhibition of colony formation at doses ranging between 0.1 and 2.5 μM in all cell lines, the HYB 508 control sequence showed a modest or no growth inhibitory effect. HYB 165 determined an inhibition of 1A9PTX22 cell growth of approximately 5% at a dose of 0.1 μM, of about 50% at 0.5 μM, of about 82% at 1 μM and achieved over 95% at 2.5 μM. Conversely, HYB 508 caused a growth inhibition which at the highest dose of 2.5 μM achieved 10%. See FIG. 5.

Conclusions: HYB 165 causes a dose-dependent growth inhibitory effect on 1A9PTX22 cells, while its mismatched control oligomer causes a modest growth inhibitory effect (no more than 10%).

### EXAMPLE 17

### Effect of HYB 165 and its Control HYB 508 on the Growth of 1A9PTX10 Human Ovarian Cancer Cells

Materials: 18-mer mixed backbone oligonucleotides (MBO) targeted against the N-terminal 8-13 codons of the human RIα regulatory subunit of PKA, synthesized by the procedure previously described were provided by Hybridon Inc., Cambridge, Mass. The antisense used had the following sequences: HYB 165, GCGUGCCTCCTCACUGGC (SEQ ID NO:4); HYB 508, GCAUGCTTCCACACAGGC (SEQ ID NO:9). HYB 165 and HYB 508 are compounds containing 2-O-methyl-modified ribonucleotide bases (bold italics) at the 5′ and 3′ ends and unmodified oligodeoxynucleotide bases in the middle. HYB 508 is a control oligonucleotide containing four mismatched nucleotides as underlined.

Cell lines: The 1A9PTX10 cell line, a paclitaxel (PTX)-resistant clone of the human ovarian carcinoma cell line 1A9, was isolated by exposing 1A9 cells to 5 ng/ml PTX in the presence of 5 μg/ml verapamil, a P glycoprotein antagonist. 1A9PTX10 cells were kindly provided by Dr. Giannakakou, NCI Bethesda, Md., USA. Cells were maintained in RPMI medium supplemented with 10% heat-inactivated FBS, 20 mM HEPES, pH 7.4, penicillin (100 UI/ml), streptomycin (100 μg/ml) and 4 mM glutamine (ICN, Irvine, UK) 15 ng/ml PTX and 5 μg/ml verapamil in a humidified atmosphere of 95% air and 5% CO₂ at 37° C. 7 days before experiments were performed, PTX and verapamil were removed from culture medium.

Soft agar growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of HYB 165 or HYB 508 every 48 hours for three times. After 12 days the cells were stained with nitroblue tetrazolium (Sigma, St. Louis, Mo.) and colonies larger than 0.05 mm were counted. Experiments were performed twice in triplicate

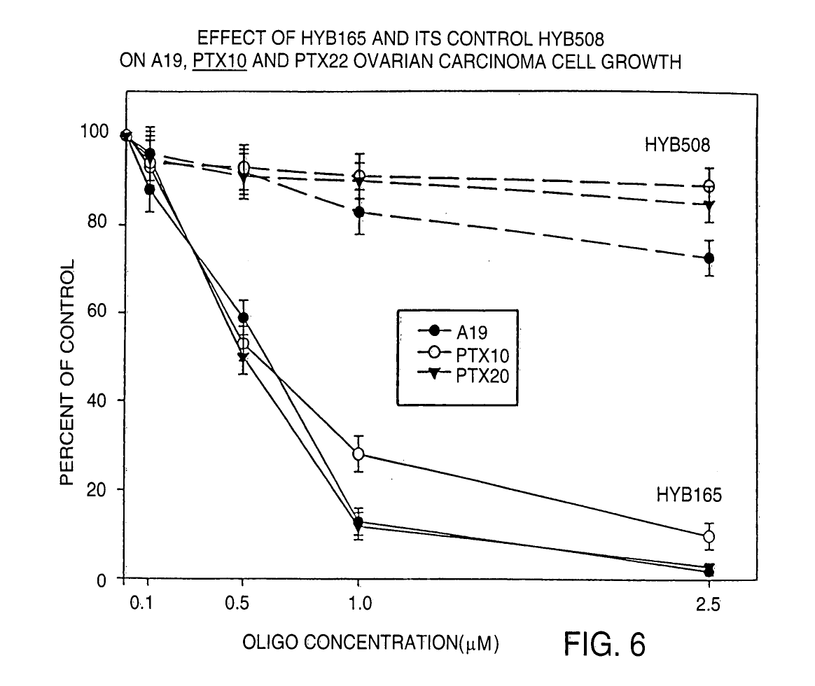

Results: Two different 18-mer MBO complementary to the RIα subunit of PKA-I sequence, HYB 165 and its control oligomer HYB 508, differing only in 4 nucleotide bases, were tested to study their effect on soft agar growth of 1A9 human ovarian cancer cells. While HYB 165 determined a dose-dependent inhibition of colony formation at doses ranging between 0.1 and 2.5 μM in all cell lines, the HYB 508 control sequence showed a modest or no growth inhibitory effect. HYB 165 determined an inhibition of 1A9PTX10 cell growth of approximately 5% at a dose of 0.1 μM, of about 43% at 0.5 μM, of about 70% at 1 μM and achieved over 85% at 2.5 μM (FIG. 2). Conversely, HYB 508 caused a growth inhibition which at the highest dose of 2.5 μM achieved 10%. See FIG. 6.

Conclusions: HYB 165 causes a dose-dependent growth inhibitory effect on 1A9PTX10 cells, while its mismatched control oligomer causes a modest growth inhibitory effect (no more than 10%).

### EXAMPLE 18

### Effect of HYB 165 and its Control HYB 508 on the Growth of 1A9 Human Ovarian Cancer Cells

Materials: 18-mer mixed backbone oligonucleotides (MBO) targeted against the N-terminal 8-13 codons of the human RIα regulatory subunit of PKA, synthesized by the procedure previously described were provided by Hybridon Inc., Cambridge, Mass. The antisense used had the following sequences: HYB 165, GCGUGCCTCCTCACUGGC (SEQ ID NO:4); HYB 508 GCAUGCTTCCACACAGGC (SEQ ID NO:9). HYB 165 and HYB 508 are compounds containing 2-O-methyl-modified ribonucleotide bases (bold italics) at the 5′ and 3′ ends and unmodified oligodeoxynucleotide bases in the middle. HYB 508 is a control oligonucleotide containing four mismatched nucleotides as underlined.

Cell Lines: The 1A9 cell line is a clone of the human ovarian carcinoma cell line, A2780. 1A9 cells were kindly provided by Giannakakou, NCI Bethesda, Md., USA. Cells were maintained in RPMI medium supplemented with 10% heat-inactivated FBS, 20 mM HEPES, pH 7.4 penicillin (100 UI/ml), streptomycin (100 μg/ml) and 4 mM glutamine (ICN, Irvine, UK) in a humidified atmosphere of 95% air and 5% CO₂ at 37° C.

Soft Agar Growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of HYB 165 or HYB 508 every 48 hours for three times. After 12 days the cells were stained with nitroblue tetrazolium (Sigma, St. Louis, Mo.) and colonies larger than 0.05 mm were counted. Experiments were performed twice in triplicate.

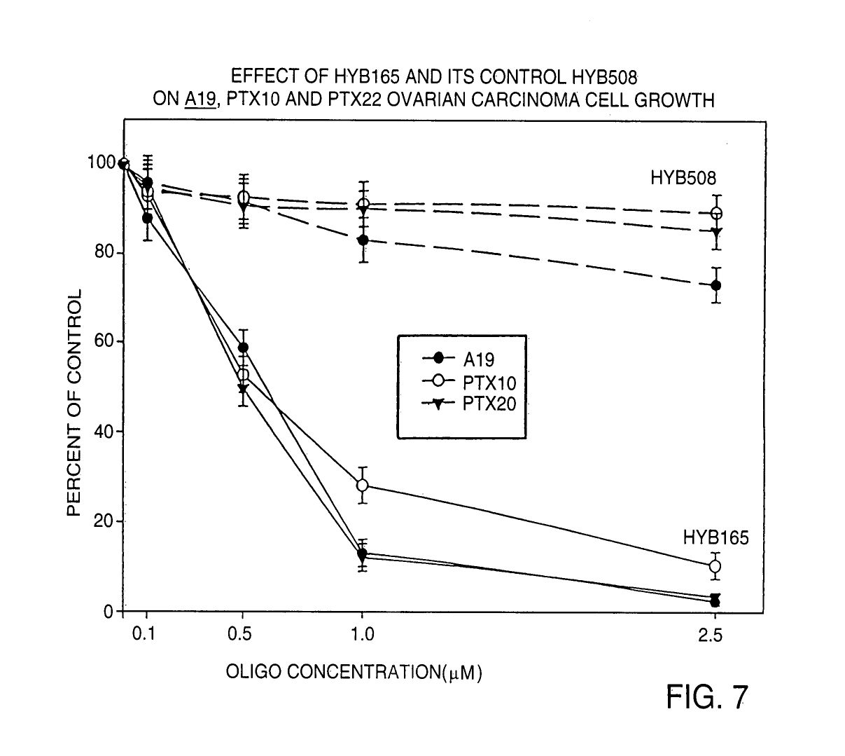

Results: Two different 18-mer MBOs complementary to the RIα subunit of PKA-I sequence, HYB 165 and its control oligomer HYB 508, differing only in 4 nucleotide bases, were studied to evaluate their effect on soft agar growth of 1A9 human ovarian cancer cells. While HYB 165 determined a dose-dependent inhibition of colony formation at doses ranging between 0.1 and 2.5 μM in all cell lines, the HYB 508 control sequence showed a modest or no growth inhibitory effect. HYB 165 determined an inhibition of 1A9 cell growth of approximately 5% at a dose of 0.1 μM, of about 41% at 0.5 μM, of about 90% at 1 μM and achieved over 95% at 2.5 μM (FIG. 2). Conversely, HYB 508 caused a growth inhibition which at the highest dose of 2.5 μM achieved 20% inhibition. See FIG. 7.

Conclusions: HYB 165 causes a dose-dependent growth inhibitory effect on 1A9 cells, while its mismatched control oligomer causes a modest growth inhibitory effect (no more than 20%).

### EXAMPLE 19

### Effect of HYB 508 with or without Monoclonal Antibody MAb C225 on the Growth of ZR-75-1 Human Breast Cancer Cells

Materials: HYB 508, a 18-mer mixed backbone oligonucleotides (MBO) targeted against the N-terminal 8-13 codons of the human RIα regulatory subunit of PKA, synthesized by the procedure previously described was provided by Hybridon Inc., Cambridge, Mass. The antisense used had the following sequence: HYB 508, GCAUGCTTCCACACAGGC (SEQ ID NO:9) and contains 2-O-methyl-modified ribonucleotide bases (bold italics) at the 5′ and 3′ ends and unmodified oligodeoxynucleotide bases in the middle. HYB 508 is a control oligonucleotide of HYB 165, containing four mismatched nucleotides (underlined). The monoclonal antibody MAb C225 is a human-mouse chimeric IgG₁ that binds to the EGFR, competes with natural ligands for receptor binding and blocks the EGFR tyrosine kinase activation. Clinical grade MAbC225 was-kindly provided by Dr. H. Waksal, ImClone Systems, New York, N.Y.

Cell Lines: ZR-75-1 human breast cancer cells were purchased from American Type Culture Collection (Rockville, Md., USA). Cells were maintained in DMEM medium supplemented with 10% heat-inactivated FBS, 20 mM HEPES, pH 7.4, penicillin (100 UI/ml), streptomycin (100 μg/ml) and 4 mM glutamine (ICN, Irvine, UK) in a humidified atmosphere of 95% air and 5% CO₂ at 37° C.

Soft Agar Growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of MAb C225 and/or of HYB 508 every 48 hours for three times. After 12 days the cells were stained with nitroblue tetrazolium (Sigma) and colonies larger than 0.05 mm were counted. Experiments were performed twice in triplicate.

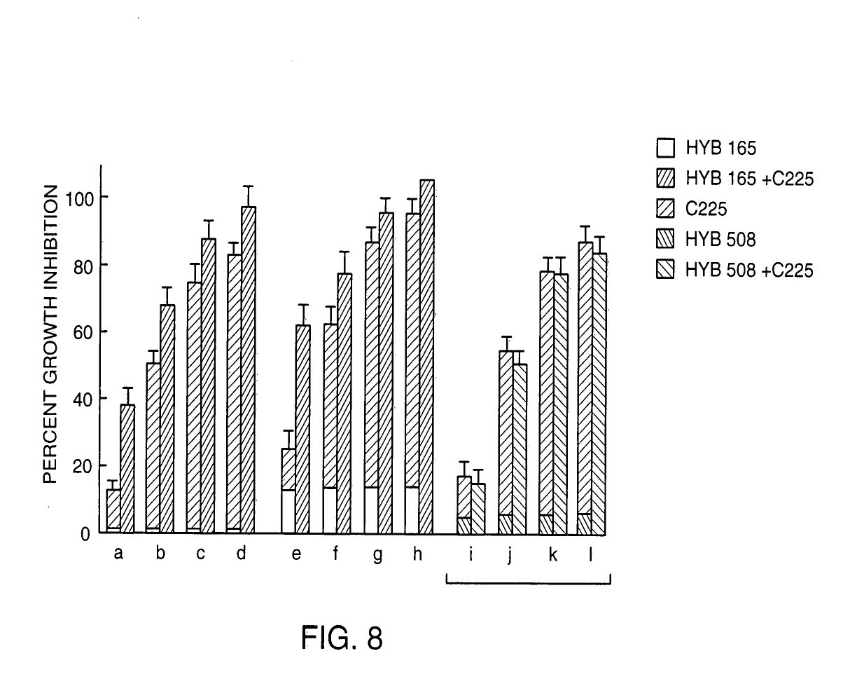

Results: HYB 508 0.5 μM (i-l), which alone causes about 5% inhibition of ZR-75-1 cell growth, was used in combination with i) MAb C225 0.25 μg/ml, which alone causes about 10% inhibition, determining an average 12% inhibition; j) MAb C225 0.5 μg/ml, which alone causes about 47% inhibition, determining an average 45% inhibition; k) MAb C225 1 μg/ml, which alone causes about 68% inhibition, determining an average 77% inhibition; l) MAb C225 2.5 μg/ml, which alone causes about 76% inhibition, determining an average 82% inhibition. See FIG. 8.

Conclusions: HYB 508 at the dose of 0.5 μM showed no cooperative antiproliferative effect on the growth of ZR-75-1 cells when used in combination with different doses of MAb C225. FIG. 8 shows the effect of the combination of two different agents on the growth of ZR-75-1 breast cancer cells. HYB 508 0.5 μM (i-l) in combination with MAb C225 0.25 μg/ml (i), 0.5 μg/ml (j), 1 μg/ml (k) and 2.5 μg/ml (l). Data are expressed as percentage growth inhibition in reference to the growth of untreated control cells. The height of the bars on the left represents the sum of the individual agents effects and the expected percentage growth inhibition if drugs are additive when used in combination. The total height of the solid bar indicates the actual observed growth inhibition when drugs were used in combination. Therefore, the differences between the heights of the paired bars reflect the magnitude of synergism of growth inhibition. The data represent means and standard errors of triplicate determination of at least two experiments.

### EXAMPLE 20

### Effect of HYB 165 and HYB 618 on the Growth of OVCAR-3 Ovarian Cancer Cells

Materials: 18-mer mixed backbone oligonucleotides (MBO) targeted against the N-terminal 8-13 codons of the human RIα regulatory subunit of PKA, synthesized by the procedure previously described were provided by Hybridon Inc., Cambridge, Mass. The antisense used had the following sequences: HYB 165, GCGUGCCTCCTCACUGGC (SEQ ID NO:4); HYB 618, GCAUGCATCCGCACAGGC (SEQ ID NO:10). HYB 165 and HYB 618 are compounds containing 2-O-methyl-modified ribonucleotide bases (bold italics) at the 5′ and 3′ ends and unmodified oligodeoxynucleotide bases in the middle. HYB 618 is a control oligonucleotide containing four mismatched nucleotides as underlined.

Cell Lines: OVCAR human ovarian cancer cells were purchased from American Type Culture Collection (Rockville, MD, USA). Cells were maintained in DMEM and HAM'S F-12 (1:1) supplemented with 10% heat-inactivated FBS, 20 mM HEPES, pH 7.4, penicillin (100 UI/ml), streptomycin (100 μg/ml) and 4 mM glutamine (ICN, Irvine, UK) in a humidified atmosphere of 95% air and 5% CO₂ at 37° C.

Soft Agar Growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of HYB 165 or HYB 295 every 48 hours for three times. After 12 days the cells were stained with nitroblue tetrazolium (Sigma, St. Louis, Mo.) and colonies larger than 0.05 mm were counted. Experiments were performed twice in triplicate.

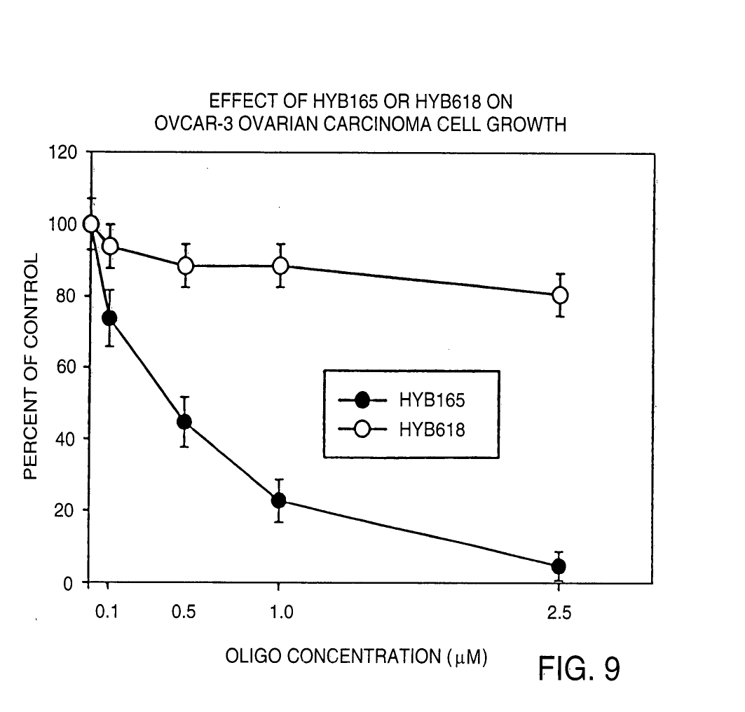

Results: Two different 18-mer MBOs complementary to the RIα subunit of PKA-I sequence, HYB 165 and its control oligomer HYB 618, differing only in 4 nucleotide bases, were tested to study their effect on soft agar growth of GEO human colon cancer cells. While HYB 165 determined a dose-dependent inhibition of colony formation at doses ranging between 0.1 and 2.5 μM in all cell lines, the HYB 618 control sequence showed a modest or no growth inhibitory effect. HYB 165 determined an inhibition of OVCAR-3 cell growth of approximately 25% at a dose of 0.1 μM, of about 58% at 0.5 μM, of about 75% at 1 μM and about 95% at 2.5 μM (FIG. 2). Conversely, HYB 618 caused a growth inhibition which at the highest dose of 2.5 μM achieved 15%. See FIG. 9.

Conclusions: HYB 165 causes a dose-dependent growth inhibitory effect on OVCAR-3 cells, while its mismatched control oligomer causes a modest growth inhibitory effect (less than 15%).

### EXAMPLE 21

### Effect of HYB 165 with or without Docetaxel on the Growth of ZR-75-1 Human Breast Cancer Cells

Materials: HYB 165, a 18-mer mixed backbone oligonucleotides (MBO) targeted against the N-terminal 8-13 codons of the human RIα regulatory subunit of PKA, synthesized by the procedure previously described was provided by Hybridon Inc., Cambridge, Mass. The antisense used had the following sequence: HYB 165, GCGUGCCTCCTCACUGGC (SEQ ID NO:4) and contains 2-O-methyl-modified ribonucleotide bases (bold italics) at the 5′ and 3′ ends and unmodified oligodeoxynucleotide bases in the middle. Docetaxel was a kind gift from Rhone Poulenc Rorer, Origgio, Italy, and used after dilution in appropriate solvent as 100x concentrated stock.

Cell Lines: ZR-75-1 human breast cancer cells were purchased from American Type Culture Collection (Rockville, Md., USA). Cells were maintained in DMEM medium supplemented with 10% heat-inactivated FBS, 20 mM HEPES, pH 7.4, penicillin (100 UI/ml), streptomycin (100 [μg/ml) and 4 mM glutamine (ICN, Irvine, UK) in a humidified atmosphere of 95% air and 5% CO₂ at 37° C.

Soft Agar Growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of docetaxel (day 0). The HYB 165 was added after 12 hrs (day 1) and on day 3. Twelve days after the last treatment, cells were stained with nitroblue tetrazolium (Sigma) and colonies larger than 0.05 mm were counted. Experiments were performed twice in triplicate.

Results: A dose-dependent effect of docetaxel at doses ranging between 0.01 and 0.3 nM was observed, determining up to about 80% growth inhibition. HYB 165 0.1(a-d) μM, which alone causes about 5% inhibition, was added to ZR-75-1 cells treated with a) docetaxel 0.01 nM, which alone causes less than 15% inhibition, determining an average 40% inhibition; b) docetaxel 0.03 nM, which alone causes about 40% inhibition, determining an average 70% inhibition; c) docetaxel 0.1 nM, which alone causes about 72% inhibition, determining an average 86% inhibition; d) docetaxel 0.3 nM, which alone causes about 85% inhibition, determining an average 97%.

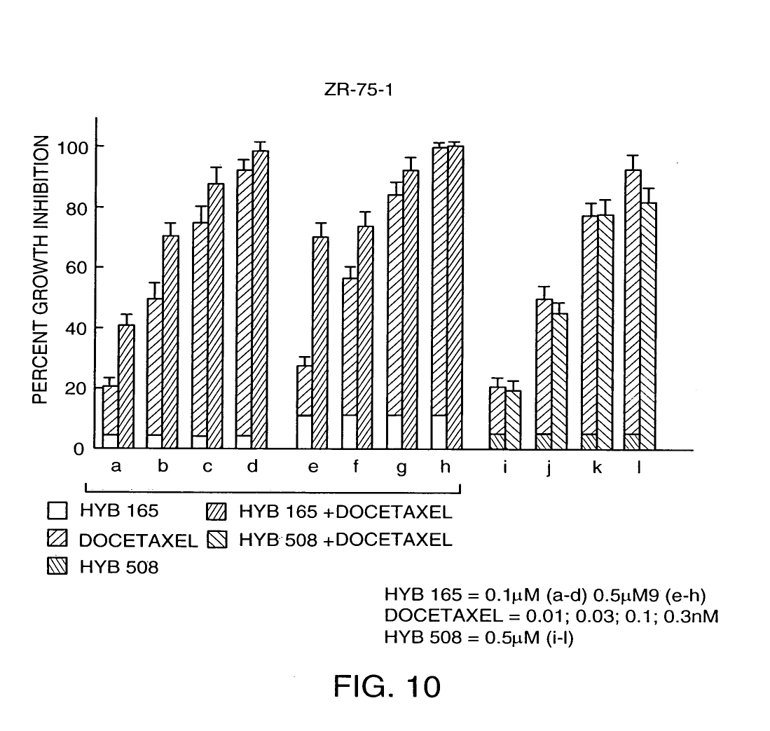

HYB 165 0.5 μM(e-f), which alone causes about 15% inhibition, was added to ZR-75-1 cells treated with e) docetaxel 0.01 nM, which alone causes less than 15% inhibition, determining an average 65% inhibition; ) docetaxel 0.03 nM, which alone causes about 40% inhibition, determining an average 66% inhibition; g) docetaxel 0.1 nM, which alone causes about 72% inhibition, determining an average 86% inhibition; h) docetaxel 0.3 nM, which alone causes about 85% inhibition, determining an average 99% inhibition. See FIG. 10.

Conclusions: HYB 165 at the low inhibitory doses of 0.1 μM and 0.5 μM cooperatively inhibits the growth of ZR-75-1 cells when used in a sequential combination with different doses of docetaxel. FIG. 10 shows the effect of the combination of two different agents on the growth of ZR-75-1 breast cancer cells. HYB 165 0.1 μM (a-d) and 0.5 μM (e-f) in combination with Docetaxel 0.01 nM (a-e); 0.03 nM (b-f); 0.1 nM (c-g); 0.3 nM (d-h). Data are expressed as percentage growth inhibition in reference to the growth of untreated control cells. The height of the bars on the left represents the sum of the individual agents effects and the expected percentage growth inhibition if drugs are additive when used in combination. The total height of the solid bar indicates the actual observed growth inhibition when drugs were used in combination. Therefore, the differences between the heights of the paired bars reflect the magnitude of synergism of growth inhibition. The data represent means and standard errors of triplicate determination of at least two experiments.

### EXAMPLE 22

### Effect of HYB 508 with or without Docetaxel on the Growth of ZR-75-1 Human Breast Cancer Cells

Materials: HYB 508, a 18-mer mixed backbone oligonucleotides (MBO) targeted against the N-terminal 8-13 codons of the human RIα regulatory subunit of PKA, synthesized by the procedure previously described was provided by Hybridon Inc., Cambridge, Mass. The antisense used had the following sequence: HYB 508, GCAUGCTTCCACACAGGC (SEQ ID NO:9) and contains 2-O-methyl-modified ribonucleotide bases (bold italics) at the 5′ and 3′ ends and unmodified oligodeoxynucleotide bases in the middle. HYB 508 is a control oligonucleotide of HYB 165, containing four mismatched nucleotides (underlined). Docetaxel was a kind gift from Rhone Poulenc Rorer, Origgio, Italy, and used after dilution in appropriate solvent as 100× concentrated stock.

Cell Lines: ZR-75-1 human breast cancer cells were purchased from American Type Culture Collection (Rockville, Md., USA). Cells were maintained in DMEM medium supplemented with 10% heat-inactivated FBS, 20 mM HEPES, pH 7.4, penicillin (100 UI/ml), streptomycin (100 μg/ml) and 4 mM glutamine (ICN, Irvine, UK) in a humidified atmosphere of 95% air and 5% CO₂ at 37° C.

Soft Agar Growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of docetaxel (day 0). The HYB 508 was added after 12 hrs and on day 2, 3 and 4. After 12 days the cells were stained with nitroblue tetrazolium (Sigma) and colonies larger than 0.05 mm were counted. Experiments were performed twice in triplicate.

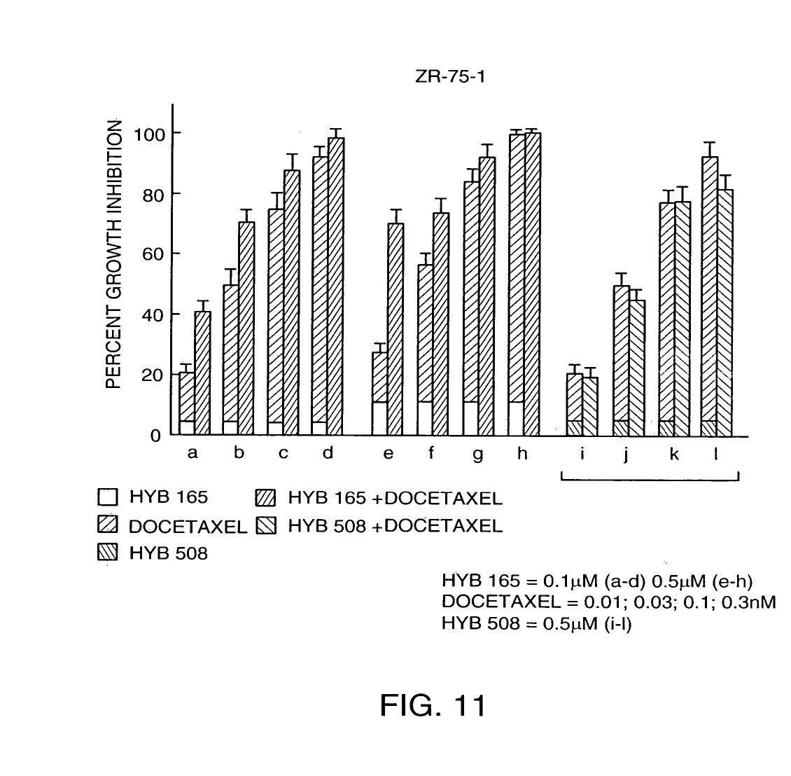

Results: A dose-dependent effect of docetaxel at doses ranging between 0.01 and 0.3 nM was observed, determining up to about 80% growth inhibition. HYB 508 0.5 μM (i-l), which alone causes about 7% inhibition, was added to ZR-75-1 cells treated with cells treated with: i) docetaxel 0.01 nM, which alone causes less than 15% inhibition, determining an average 20% inhibition; j) docetaxel 0.03 nM, which alone causes about 40% inhibition, determining an average 42% inhibition; k) docetaxel 0.1 nM, which alone causes about 72% inhibition, determining an average 78% inhibition; l) docetaxel 0.3 nM, which alone causes about 85% inhibition, determining an average 82%. See FIG. 11.

Conclusions: HYB 508 at the dose of 0.5 μM showed no cooperative antiproliferative effect on the growth of ZR-75-1 cells when used in a sequential combination with different doses of docetaxel. FIG. 11 shows the effect of the combination of two different agents on the growth of ZR-75-1 breast cancer cells. HYB 508 0.5 μM (i-l) in combination with Docetaxel 0.01 nM (i); 0.03 nM (j); 0.1 nM (k); 0.3 nM (1). Data are expressed as percentage growth inhibition in reference to the growth of untreated control cells. The height of the bars on the left represents the sum of the individual agents effects and the expected percentage growth inhibition if drugs are additive when used in combination. The total height of the solid bar indicates the actual observed growth inhibition when drugs were used in combination. Therefore, the differences between the heights of the paired bars reflect the magnitude of synergism of growth inhibition. The data represent means and standard errors of triplicate determination of at least two experiments.

### EXAMPLE 23

### Effect of HYB 165 With or Without Monoclonal Antibody MAb C225 on the Growth of ZR-75-1 Human Breast Cancer Cells

Materials: HYB 165, a 18-mer mixed backbone oligonucleotides (MBO) targeted against the N-terminal 8-13 codons of the human RIα regulatory subunit of PKA, synthesized by the procedure previously described was provided by Hybridon Inc., Cambridge, MA. The antisense used had the following sequence: HYB 165, GCGUGCCTCCTCACUGGC (SEQ ID NO:4) and contains 2-O-methyl-modified ribonucleotide bases (bold italics) at the 5′ and 3′ ends and unmodified oligodeoxynucleotide bases in the middle. The monoclonal antibody MAb C225 is a human-mouse chimeric IgG₁ that binds to the EGFR, competes with natural ligands for receptor binding and blocks the EGFR tyrosine kinase activation. Clinical grade MAb C225 was kindly provided by Dr. H. Waksal, ImClone Systems, New York, N.Y.

Cell Lines: ZR-75-1 human breast cancer cells were purchased from American Type Culture Collection (Rockville, Md., USA). Cells were maintained in DMEM medium supplemented with 10% heat-inactivated FBS, 20 mM HEPES, pH 7.4 penicillin (100 UI/ml), streptomycin (100 μg/ml) and 4 mM glutamine (ICN, Irvine, UK) in a humidified atmosphere of 95% air and 5% CO₂ at 37° C.

Soft Agar Growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of MAb C225 and/or of HYB 165 every 48 hours for three times. After 12 days the cells were stained with nitroblue tetrazolium (Sigma) and colonies larger than 0.05 mm were counted. Experiments were performed twice in triplicate.

Results: HYB 165 0.1 μM (a-d), which alone causes about 2% inhibition of ZR-75-1 cell growth, was used in combination with a) MAb C225 0.25 μg/ml, which alone causes about 10% inhibition, determining an average 37% inhibition; b) MAb C225 0.5 μg/ml, which alone causes about 47% inhibition, determining an average 65% inhibition; c) MAb C225 1 μg/ml, which alone causes about 68% inhibition, determining an average 85% inhibition; d) MAb C225 2.5 μg/ml, which alone causes about 76% inhibition, determining an average 90% inhibition.

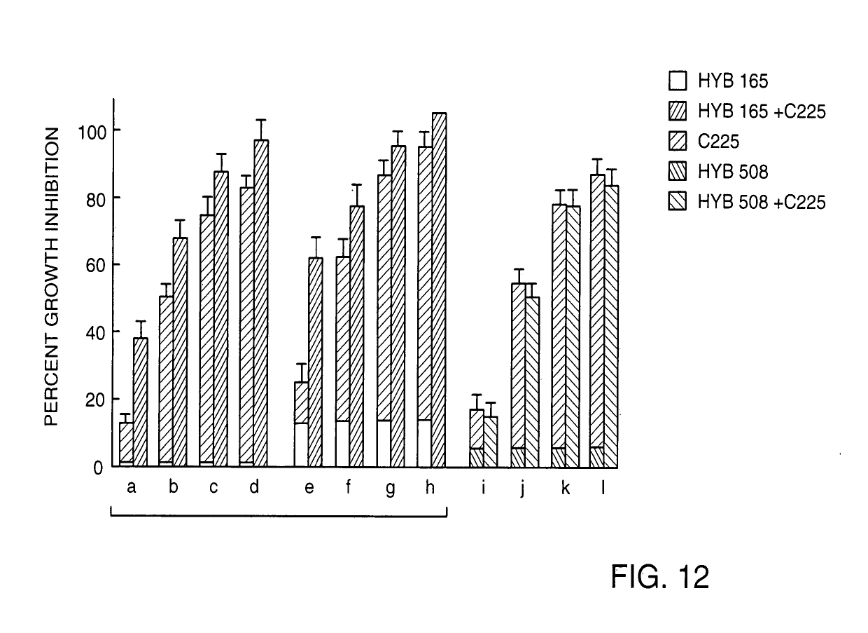

HYB 165 at the higher dose of 0.5 μM (e-h), which alone causes about 10% inhibition of ZR-75-1 cell growth, was used in combination with e) MAb C225 0.25 μg/ml, which alone causes about 10% inhibition, determining an average 57% inhibition; f) MAb C225 0.5 μg/ml, which alone causes about 47% inhibition, determining an average 70% inhibition; g) MAb C225 1 μg/ml, which alone causes about 68% inhibition, determining an average 90% inhibition; h) MAb C225 2.5 μg/ml, which alone causes about 76% inhibition, determining an average 98% inhibition. See FIG. 12.

Conclusions: HYB 165 at the low inhibitory dose of 0.1 I M and 0.5 μM cooperatively inhibit the growth of ZR-75-1 cells when used in combination with different doses of MAb C225. FIG. 12 shows the effect of the combination of two different agents on the growth of ZR-75-1 breast cancer cells. HYB 165 0.1 μM (a-d) and 0.5 μM (e-f) or HYB 508 0.5 μM (i-l) in combination with MAb C225 0.25 μg/ml (a,e,i), 0.5 μg/ml (b,f j), 1 μg/ml (c,g,k) and 2.5 μg/ml (d,h,l). Data are expressed as percentage growth inhibition in reference to the growth of untreated control cells. The height of the bars on the left represents the sum of the individual agents effects and the expected percentage growth inhibition if drugs are additive when used in combination. The total height of the solid bar indicates the actual observed growth inhibition when drugs were used in combination. Therefore, the differences between the heights of the paired bars reflect the magnitude of synergism of growth inhibition. The data represent means and standard errors of triplicate determination of two experiments.

### EXAMPLE 24

### Effect of HYB 165 and HYB 295 on the Growth of ZR-75-1 Human Breast Cancer Cells

Materials: 18-mer mixed backbone oligonucleotides (MBO) targeted against the N-terminal 8-13 codons of the human RIα regulatory subunit of PKA, synthesized by the procedure previously described were provided by Hybridon Inc., Cambridge, Mass. The antisense used had the following sequences: HYB 165, GCGUGCCTCCTCACUGGC (SEQ ID NO:4); HYB 295, GCAUGCATCCGCACAGGC (SEQ ID NO:10). HYB 165 and HYB 295 are compounds containing 2-O-methyl-modified ribonucleotide bases (bold italics) at the 5′ and 3′ ends and unmodified oligodeoxynucleotide bases in the middle. HYB 295 is a control oligonucleotide containing four mismatched nucleotides as underlined.

Cell Lines: ZR-75-1 human breast cancer cells were purchased from American Type Culture Collection (Rockville, Md., USA). Cells were maintained in DMEM medium supplemented with 10% heat-inactivated FBS, 20 mM HEPES, pH 7.4, penicillin (100 UI/ml), streptomycin (100 μg/ml) and 4 mM glutamine (ICN, Irvine, UK) in a humidified atmosphere of 95% air and 5% CO₂ at 37° C.

Soft Agar Growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of HYB 165 or HYB 295 every 48 hours for three times. After 12 days the cells were stained with nitroblue tetrazolium (Sigma, St. Louis, Mo.) and colonies larger than 0.05 mm were counted. Experiments were performed twice in triplicate.

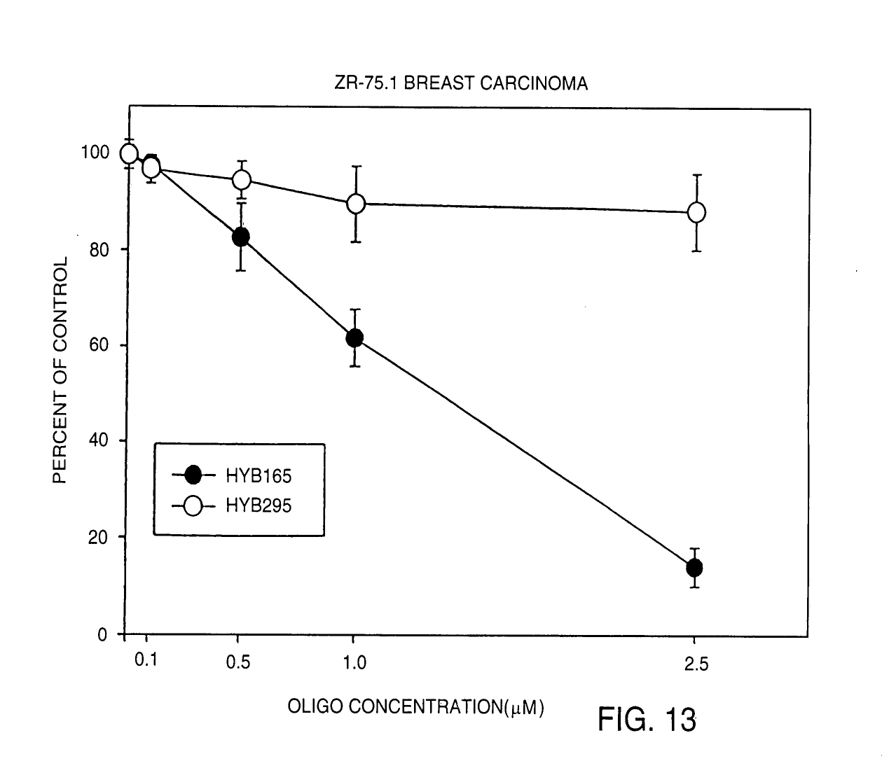

Results: Two different 18-mer MBOs complementary to the RIα subunit of PKA-I sequence, HYB 165 and its control oligomer HYB 295, differing only in 4 nucleotide bases, were tested to study their effect on soft agar growth of ZR-75-1 human breast cancer cells. While HYB 165 determined a dose-dependent inhibition of colony formation at doses ranging between 0.1 and 2.5 μM in all cell lines, the HYB 295 control sequence showed a modest or no growth inhibitory effect. HYB 165 determined an inhibition of ZR-75-1 cell growth of approximately 5% at a dose of 0.1 μM, of about 34% at 1 μM and achieved over 85% at 2.5 μM. Conversely, HYB 295 caused a growth inhibition which at the highest dose of 2.5 μM achieved 10%. See FIG. 13.

Conclusions: HYB 165 causes a dose-dependent growth inhibitory effect on ZR-75-1 cells, while its mismatched control oligomer causes a modest growth inhibitory effect (no more than 10%).

### EXAMPLE 25

### Effect of HYB 165 and HYB 508 on the Growth of ZR-75-1 Human Breast Cancer Cells

Materials: 18-mer mixed backbone oligonucleotides (MBO) targeted against the N-terminal 8-13 codons of the human RIα regulatory subunit of PKA, synthesized by the procedure previously described were provided by Hybridon Inc., Cambridge, Mass. The antisense used had the following sequences: HYB 165, GCGUGCCTCCTCACUGGC (SEQ ID NO:4); HYB 508, GCAUGCTTCCACACAGGC (SEQ ID NO:9). HYB 165 and HYB 508 are compounds containing 2-O-methyl-modified ribonucleotide bases (bold italics) at the 5′ and 3′ ends and unmodified oligodeoxynucleotide bases in the middle. HYB 508 is a control oligonucleotide containing four mismatched nucleotides as underlined.

Cell Lines: ZR-75-1 human breast cancer cells were purchased from American Type Culture Collection (Rockville, Md., USA). Cells were maintained in DMEM medium supplemented with 10% heat-inactivated FBS, 20 mM HEPES, pH 7.4, penicillin (100 UI/ml), streptomycin (100 μg/ml) and 4 mM glutamine (ICN, Irvine, UK) in a humidified atmosphere of 95% air and 5% CO₂ at 37° C.

Soft Agar Growth. Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of HYB 165 or HYB 508 every 48 hours for three times. After 12 days the cells were stained with nitroblue tetrazolium (Sigma, St. Louis, Mo.) and colonies larger than 0.05 mm were counted. Experiments were performed twice in triplicate.

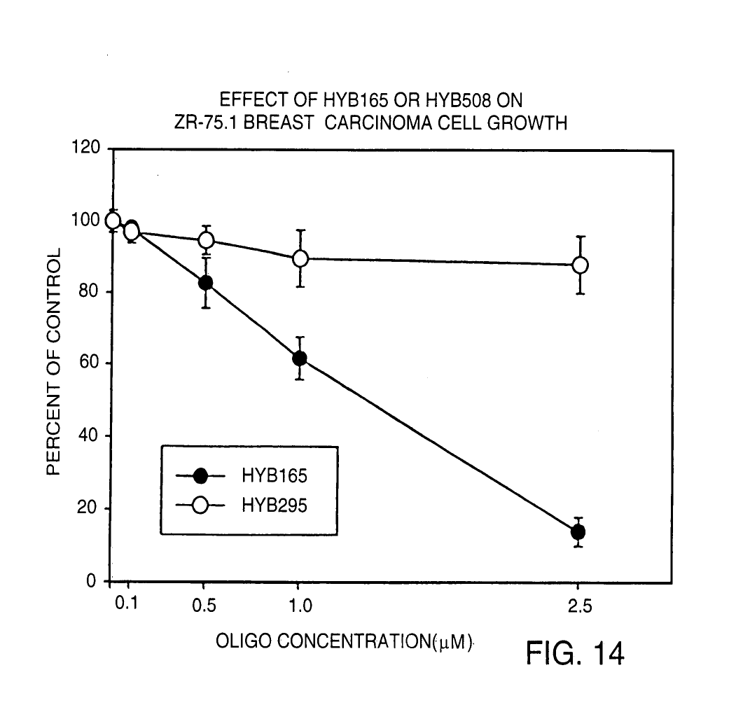

Results: Two different 18-mer MBOs complementary to the RIα subunit of PKA-I sequence, HYB 165 and its control oligomer HYB 508, differing only in 4 nucleotide bases, were tested to study their effect on soft agar growth of ZR-75-1 human breast cancer cells. While HYB 165 determined a dose-dependent inhibition of colony formation at doses ranging between 0.1 and 2.5 μM in all cell lines, the HYB 508 control sequence showed a modest or no growth inhibitory effect. HYB 165 determined an inhibition of ZR-75-1 cell growth of approximately 5% at a dose of 0.1 μM, of about 34% at 1 μM and achieved over 85% at 2.5 μM. Conversely, HYB 508 caused a growth inhibition which at the highest dose of 2.5 μM achieved 10%. See FIG. 14.

Conclusions: HYB 165 causes a dose-dependent growth inhibitory effect on ZR-75-1 cells, while its mismatched control oligomer causes a modest growth inhibitory effect (no more than 10%).

### EXAMPLE 26

### Effect of HYB 165 and HYB 295 on the Growth of GEO Colon Cancer Cells

Materials: 18-mer mixed backbone oligonucleotides (MBO), targeted against the N-terminal 8-13 codons of the human RIα regulatory subunit of PKA, synthesized by the procedure previously described were provided by Hybridon Inc., Cambridge, Mass. The antisense used had the following sequences: HYB 165, GCGUGCCTCCTCACUGGC (SEQ ID NO:4); HYB 295, GCAUGCATCCGCACAGGC (SEQ ID NO:10). HYB 165 and HYB 295 are compounds containing 2-O-methyl-modified ribonucleotide bases (bold italics) at the 5′ and 3′ ends and unmodified oligodeoxynucleotide bases in the middle. HYB 295 is a control oligonucleotide containing four mismatched nucleotides as underlined.

Cell Lines: GEO human colon cancer cells were purchased from American Type Culture Collection (Rockville, Md., USA). Cells were maintained in McCoy's Medium 5A supplemented with 10% heat-inactivated FBS, 20 mM HEPES, pH 7.4, penicillin (100 UI/ml), streptomycin (100 μg/ml) and 4 mM glutamine (ICN, Irvine, UK) in a humidified atmosphere of 95% air and 5% CO₂ at 37° C.

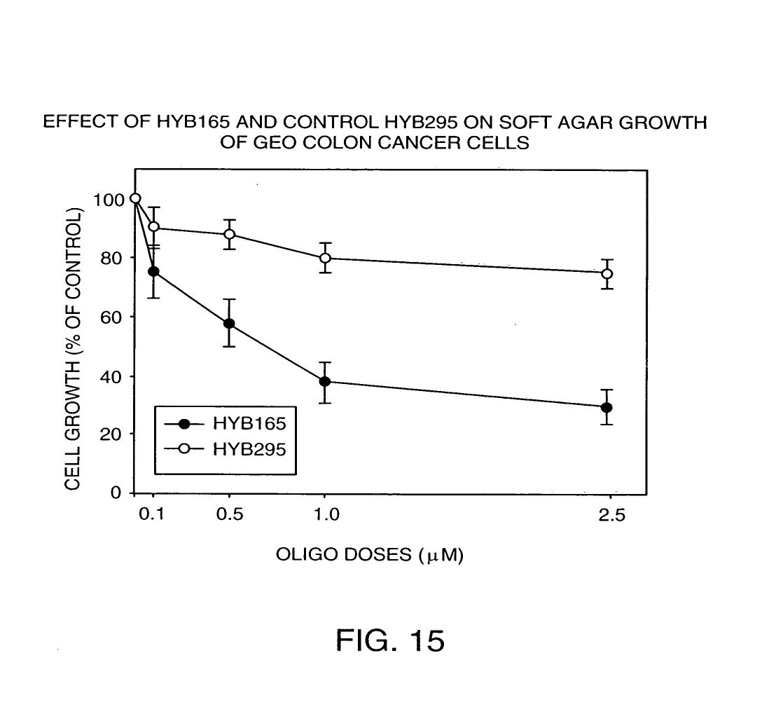

Soft Agar Growth: Cells (10⁴ cells/well) were seeded in 0.5 ml of 0.3% Difco Noble agar (Difco, Detroit, Mich.) supplemented with complete culture medium. This suspension was layered over 0.5 ml of 0.8% agar-medium base layer in 24 multiwell cluster dishes (Becton Dickinson) and treated with various concentrations of HYB 165 or HYB 295 every 48 hours for three times. After 12 days the cells were stained with nitroblue tetrazolium (Sigma, St. Louis, Mo.) and colonies larger than 0.05 mm were counted. See FIG. 15. Experiments were performed twice in triplicate.

### EXAMPLE 27

### HYB 165 Inhibits Tumor Growth after I.P. or Oral Administration

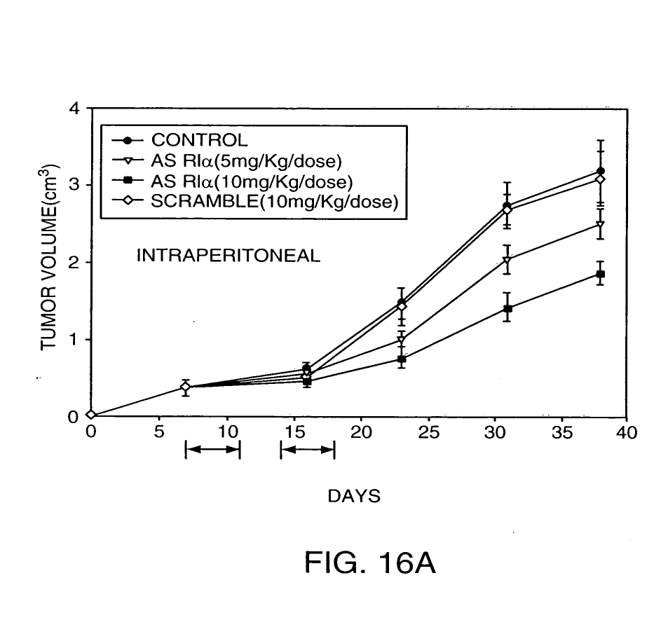

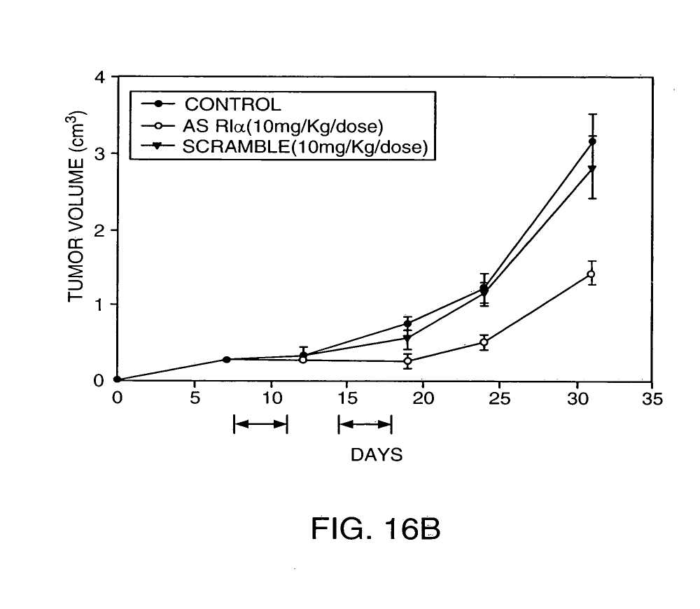

We investigated the antitumor activity of HYB 165 (AS RIα) in nude mice bearing GEO colon cancer xenografts, using either the intraperitoneal (i.p.) or the oral route of administration. When established GEO tumors of approximately 0.2 cm³ were detectable, groups of 10 mice were treated i.p. with either HYB 165 or a control modified backbone oligonucleotide with a scrambled sequence, at 5 or 10 mg/kg/dose, daily on days 7 to 11 and 14 to 18. FIG. 16A shows that i.p. administration of HYB 165 caused a dose-dependent inhibition of growth up to 40% at a dose of 10 mg/kg/dose. The control oligonucleotide produced no inhibition at 10 mg/kg/dose.

Following oral administration, modified backbone oligonucleotides (MBOs) are absorbed in the upper and lower part of the GI tract and distributed to major organs (S. Agrawal and R. Zhang, In: Antisense Research and Application, S. T. Crooke, ed.), Handbook of Experimental Pharmacology, Springer, Berlin, p. 525-543 (1998). Therefore, HYB 165 and the control oligonucleotide were administered to GEO tumor-bearing mice as described above, except that HYB 165 and the control oligonucleotide were administered orally. As shown in FIG. 16B, at a dose of 10 mg/kg/dose, the two cycles of treatment with HYB 165 caused an average inhibition of tumor growth of about 60% as compared to untreated mice, while the tumor size of the mice treated with the control scramble oligonucleotide was only slightly affected.

### EXAMPLE 28

### Oral HYB 165 Cooperatively Inhibits Tumor Growth and Increases Survival in Combination with Taxol

On day 7 after tumor cell injection, one group of 10 mice was treated with taxol (20 mg/kg/dose, i.p.), and the treatment was repeated every 2 weeks (on day 21 and day 35) for a total of three cycles. Two other groups of mice were treated with either HYB 165 (AS RIα) or a control MBO with a scrambled sequence (10 mg/kg/dose, p.o.), daily for five days (days 8-12). Treatment was repeated every 2 weeks (days 22-26 and days 36-40) for a total of three cycles. Two more groups of mice were treated with taxol and either HYB 165 or the control MBO, administering the taxol (20 mg/kg/dose, i.p.) on day 7, followed by oral administration of either HYB 165 or the control MBO daily for five days (days 8-12). The sequential treatment was repeated with the same schedule every 2 weeks for a total of three cycles.

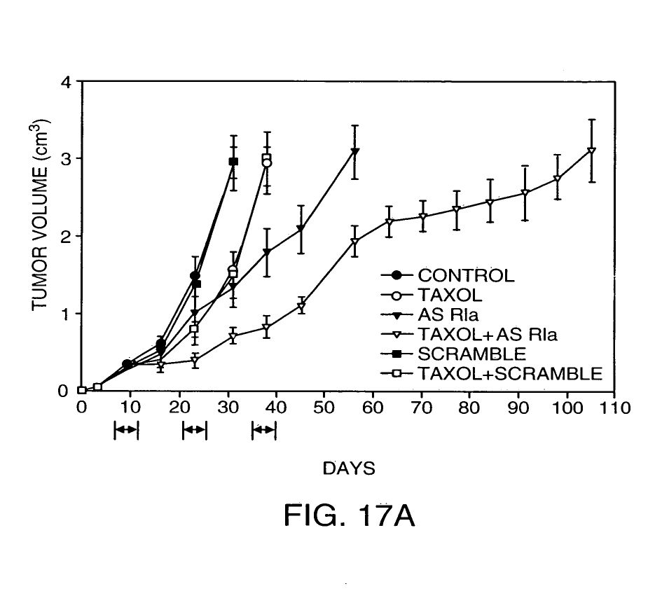

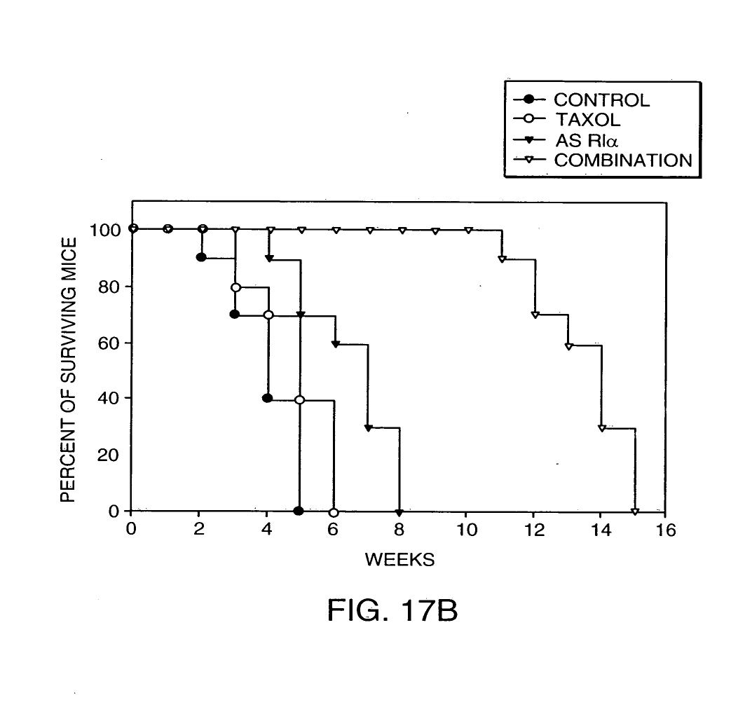

As illustrated in FIG. 17A, treatment with either taxol or the HYB 165 alone inhibited tumor growth as compared to control untreated mice or to mice treated with the scramble MBO. HYB 165 was more effective than taxol, causing over 50% inhibition of tumor size at the completion of the three cycles of treatment. However, shortly after the end of treatment, GEO tumors resumed the growth rate of those in untreated mice or in mice treated with the scramble MBO. When taxol and HYB 165 were used in combination, a marked and sustained inhibition of tumor growth was observed. In fact, tumors of mice treated with taxol and HYB 165 grew very slowly for approximately 60 days following the end of treatment, at which time they resumed a faster growth rate (FIG. 17A). Administration of the scramble MBO in combination with taxol produced an effect similar to that of taxol alone. Within approximately 5 weeks, GEO tumors reached a size not compatible with normal life in all untreated mice and in mice treated with the scramble MBO (FIG. 17B). A slight increase in survival time was observed in the group treated with taxol alone, an effect similar to that observed in mice treated with taxol followed by the scramble MBO (data not shown). Treatment with HYB 165 alone also increased survival time as compared to the control group. The delayed GEO tumor growth observed in the group treated with taxol in combination with HYB 165 was accompanied by a prolonged mouse life span, when analyzed with the log-rank test (N. Mantel, Cancer Chem. Rep., 163-170 (1966)), was significantly different as compared to controls (P<0.0001), to the taxol-treated group (P<0.0001) or to the group treated with scramble MBO plus taxol (P<0.0001). In fact, the only mice still alive at 10 weeks after tumor cell injection were those treated with the combination of taxol and HYB 165. Furthermore, about 50% of the mice in this group were still alive after 15 weeks. The combined treatment with taxol and HYB 165 was well tolerated, since no weight loss or other signs of acute or delayed toxicity were observed.

### EXAMPLE 29

### Cooperative Antitumor Effect of HYB 165 with Taxol is Accompanied by Inhibition of New Vessel Formation and Growth Factor Production

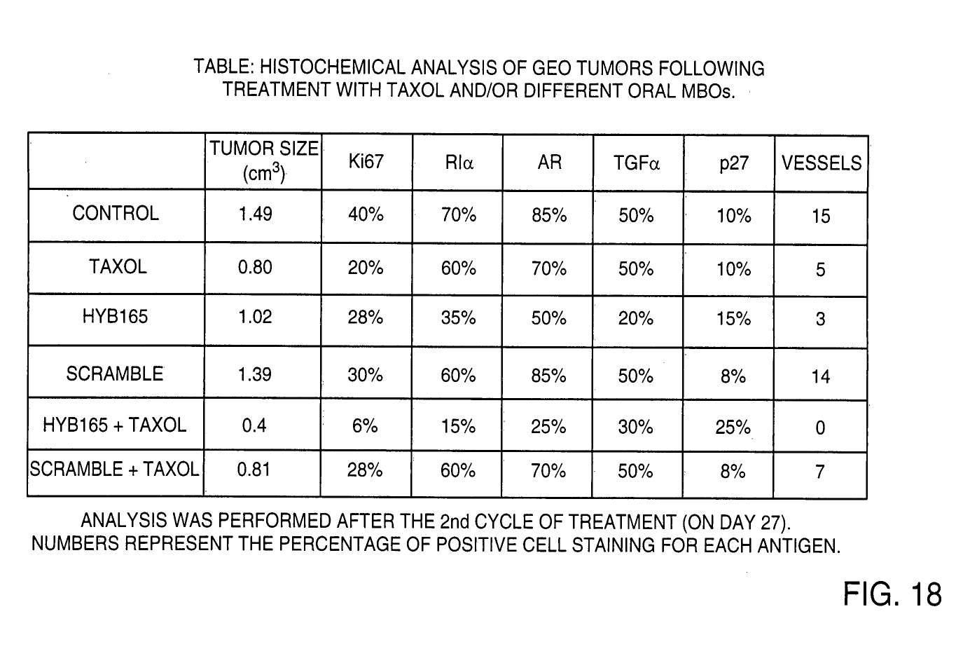

Tumor specimens from the different groups of mice were examined by histochemical analysis at different time points to evaluate the expression of a variety of biological parameters. Results of the analysis performed on tumor specimens after two cycles of treatment are presented in FIG. 18. Treatment with HYB 165 inhibited expression of the target RIα protein in the tumor. This effect was further increased when HYB 165 was used in combination with taxol. No other treatment was able to affect RIα expression. These results suggest that inhibition of RIα expression is not dependent on growth inhibition.

TGFα and AR are growth factors which bind to EGFR and control human colon cancer growth through autocrine and paracrine mechanisms (F. Ciardiello and G. Tortora, Clin. Cancer Res. 4:821-828 (1998); D. S. Salomon, Crit. Rev. Oncol. Hematol. 19:183-232 (1995)). Unlike taxol, treatment with HYB 165 inhibited the expression of TGFα and AR. Inhibition of AR was further enhanced when taxol was used in combination with HYB 165. Moreover, the combination of taxol and HYB 165 almost completely suppressed cell proliferation, as demonstrated by Ki67 staining.

Loda et al. (Nature Medicine 3:231-234 (1997)) discloses that the cyclin-dependent kinase (CDK) inhibitor p27 is directly related to cell entry into S phase and proliferation and that reduction of its expression correlates with poor prognosis in colon cancer patients. Unlike taxol, HYB 165 alone is able to increase p27 expression. Moreover, a 2.5-fold increase in intensely positive cell staining for p27 was observed in the tumor samples from mice treated with taxol and antisense RIα.

In recent years, the critical role of tumor-induced neovascularization in neoplastic development, progression and metastasis has been elucidated (J. I. Folknan, In: J. Mendelsohn et al., eds., The Molecular Basis of Cancer, pp 206-232, Philadelphia: W B Saunders (1995)). A reliable histologic estimate of novel blood vessels on tumor specimens is the microvessel count (MVC) in the most intense areas of neovascularization. In the present study, tumor-induced neovascularization was quantified by immunohistochemistry using an anti-Factor VIII related antigen monoclonal antibody (N. Weidner, Breast Cancer Res. Treat., 36:169-180 (1995)). As shown in FIG. 18, a significant inhibition of staining was obtained with HYB 165 (about 80%) as well as with taxol (over 60%), as compared to samples from untreated mice or mice treated with the scramble MBO. Combined treatment with taxol and HYB 165 completely suppressed vessel formation in GEO tumors, demonstrating that the cooperative antitumor effect was associated with the marked inhibition of several factors controlling cell cycle, proliferation and angiogenesis of this human colon cancer model.

### EXAMPLE 30

### Treatment of Colon Cancer Tumor-Bearing Mice

Female NCr-nude mice, 6-8 weeks of age, are fed ad libitum water and an autoclaved standard rodent diet. Mice are housed in microisolators on a 12 hour light cycle at 22° C. in 40-60% humidity. Mice are implanted subcutaneously in the flank with 1 mm³ HCT-1 16 human colon carcinoma fragments in the flank. Tumors are monitored twice weekly initially, then daily as the tumors reached approximately 100 mg in weight. When the tumors reach a weight between 40 mg to 221 mg (calculated weight), the animals are pair-matched into the various treatment groups. Estimated tumor weight is determined according to the equation:

$$tumor weight = \frac{{w}^{2} \times l}{2}$$

where w=width and l=length in mm of a HCT-116 tumor. Oligonucleotides complementary to PKA RIα are prepared according to standard procedures and dissolved in neutral buffered saline.

Animals are pair-matched on Day 1 into groups with 9 mice per group. The antisense oligonucleotides used are: HYB 165, GCGUGCCTCCTCACUGGC (SEQ ID NO:4); HYB 508, GCAUGCTTCCACACAGGC (SEQ ID NO:9). HYB 165 and HYB 508 contain 2-O-methyl-modified ribonucleotide bases (bold italics) at the 5′ and 3′ ends and unmodified oligodeoxynucleotide bases in the middle. HYB 508 is a control oligonucleotide containing four mismatched nucleotides as underlined.

The oligonucleotides are administered i.p. at 10 mg/kg doses on a 5/2/5/2/5/2/5 schedule (i.e., five days dosing, two days rest, repeat). Camptosar® is administered i.v. at doses of 25 or 50 mg/kg once a week for 3 weeks. For combined treatments, 5 or 10 mg/kg of the oligonucleotide is administered i.p. with Camptosar® at 25 mg/kg, or 10 mg/kg of the oligonucleotides are administered i.p. with 50 mg/kg Camptosar®. Of course, other cytotoxic drugs as described herein can be used instead of Camptosar®. Control animals are treated with vehicle i.p. on a 5/2/5/2/5/2/5 schedule. The study is terminated on day 56.

Results are determined using the tumor growth delay (TGD) endpoint method. Each mouse is euthanized when its HCT-116 tumor reaches a weight of 1.5 g; this is taken as a cancer death. Mean Day of Survival (MDS) is calculated for each group based upon the calculated day of death according to: Time to end point (calculated)=

Time to exceed endpoint (observed)—Wt₂−endpoint weight

$$\frac{{Wt}_{2} - {Wt}_{1}}{{D}_{2} - {D}_{1}}$$

where Time to exceed endpoint (observed) is the number of days it takes for each tumor to grow past the endpoint (cut-off) weight (mouse is euthanized), D₂ is the day that the mouse is euthanized, D₁, is the last day of caliper measurement before the tumor reaches endpoint, Wt₂ is tumor weight (mg) on D₂, Wt₁ is tumor weight (mg) on D₁, and endpoint weight is the predetermined “cut-off” tumor weight for the model being used. For statistical analysis, the unpaired t-test and Mann-Whitney U test (analyzing means and medians respectively) are used to determine the statistical significance of differences in survival times between groups. These analyses are conducted at a p level of 0.05 (two-tailed).

The same experiment is performed using an inverted hybrid oligonucleotide HYB 166 (SEQ ID NO:6) and its mismatched control HYB 169 (SEQ ID NO:7), which each have six deoxynucleotides at the 5′ end and seven deoxynucleotides at the 3′ end. The same experiment is also performed using an inverted chimeric oligonucleotide HYB 190 (SEQ ID NO: 1) and its mismatched control HYB 191 (SEQ ID NO:2), which each have six methylphosphonate internucleotide linkages at the center of the oligonucleotide. Other oligonucleotides and mismatched control oligonucleotides as described herein can also be used.

These results are expected to demonstrate that hybrid, inverted hybrid, and inverted chimeric antisense oligonucleotides complementary to PKA RIα can potentiate the activity of Camptosar® efficacy in a statistically significant and dose-dependent manner.

It is expected that this effect may arise from an antisense effect of the oligonucleotides on expression of the PKA RIα gene to which it is complementary. However, it is also expected that when an antisense oligonucleotide complementary to PKA RIα is used in combination with a prodrug such as Camptosar®, the oligonucleotide will exert both sequence specific as well as non-sequence specific potentiation.

### EXAMPLE 31

### Effect of Timing and Route of Oligonucleotide Administration

The study of Example 30 is repeated, but the oligonucleotide is administered initially on day 1 and Camptosar® is not administered initially until day 3. This schedule of administration is expected to be even more effective. Also, the study of Example 30 is repeated, but the oligonucleotide is administered orally. This route of administration is expected to be equally effective.

### Equivalents

Those skilled in the art will recognize, or be able to ascertain, using no more than routine experimentation, numerous equivalents to the specific substances and procedures described herein. Such equivalents are considered to be within the scope of this invention, and are covered by the following claims.

1. A method for inhibiting proliferation of cancer cells comprising:
(a) administering to the cells a first therapeutic agent comprising a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) administering to the cells a second therapeutic agent comprising a topoisomerase I inhibitor,
wherein the administering steps may be performed simultaneously or sequentially in any order.

2. The method of claim 1, wherein the oligonucleotide is a hybrid oligonucleotide.

3. The method of claim 2, wherein the oligonucleotide has a nucleotide sequence consisting of the nucleotide sequence set forth in SEQ ID NO:4.

4. The method of claim 1, wherein the oligonucleotide is an inverted hybrid oligonucleotide.

5. The method of claim 4, wherein the oligonucleotide has a nucleotide sequence consisting of the nucleotide sequence set forth in SEQ ID NO:6.

6. The method of claim 1, wherein the oligonucleotide is an inverted chimeric oligonucleotide.

7. The method of claim 5, wherein the oligonucleotide has a nucleotide sequence consisting of the nucleotide sequence set forth in SEQ ID NO: 1.

8. The method of claim 1, wherein the oligonucleotide further comprises a 2′-O-substituted nucleotide.

9. The method of claim 1, wherein the second therapeutic agent is administered prior to administration of the first therapeutic agent.

10. The method of claim 1, wherein the cancer cells are human cancer cells.

11. The method of claim 10, wherein the human cancer cells are selected from the group consisting of breast cancer cells, colon cancer cells, and ovarian cancer cells.

12. The method of claim 1, wherein the topoisomerase I inhibitor is CPT-11.

13. A pharmaceutical composition comprising:
(a) a first therapeutic agent comprising a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) a second therapeutic agent comprising a topoisomerase I inhibitor.

14. The pharmaceutical composition of claim 13, wherein the oligonucleotide is a hybrid oligonucleotide.

15. The pharmaceutical composition of claim 14, wherein the oligonucleotide has a nucleotide sequence consisting of the nucleotide sequence set forth in SEQ ID NO:4.

16. The pharmaceutical composition of claim 13, wherein the oligonucleotide is an inverted hybrid oligonucleotide.

17. The pharmaceutical composition of claim 16, wherein the oligonucleotide has a nucleotide sequence consisting of the nucleotide sequence set forth in SEQ ID NO:6.

18. The pharmaceutical composition of claim 13, wherein the oligonucleotide is an inverted chimeric oligonucleotide.

19. The pharmaceutical composition of claim 18, wherein the oligonucleotide has a nucleotide sequence consisting of the nucleotide sequence set forth in SEQ ID NO:1.

20. The pharmaceutical composition of claim 13, wherein the oligonucleotide further comprises a 2′-O-substituted nucleotide.

21. The pharmaceutical composition of claim 13, wherein the topoisomerase I inhibitor is CPT-11.

22. A method for treating cancer in an afflicted subject comprising:
(a) administering to the subject a first therapeutic agent comprising a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) administering to the subject a second therapeutic agent comprising a topoisomerase I inhibitor,
wherein the administering steps may be performed simultaneously or sequentially in any order.

23. The method of claim 22, wherein the second therapeutic agent is administered prior to administration of the first therapeutic agent.

24. The method of claim 22, wherein the subject is a human.

25. The method of claim 24, wherein the human has a cancer selected from the group consisting of breast cancer, colon cancer, and ovarian cancer.

26. The method of claim 22, wherein the oligonucleotide is a hybrid oligonucleotide.

27. The method of claim 26, wherein the oligonucleotide has a nucleotide sequence consisting of the nucleotide sequence set forth in SEQ ID NO:4.

28. The method of claim 22, wherein the oligonucleotide is an inverted hybrid oligonucleotide.

29. The method of claim 28, wherein the oligonucleotide has a nucleotide sequence consisting of the nucleotide sequence set forth in SEQ ID NO:6.

30. The method of claim 22, wherein the oligonucleotide is an inverted chimeric oligonucleotide.

31. The method of claim 30, wherein the oligonucleotide has a nucleotide sequence consisting of the nucleotide sequence set forth in SEQ ID NO:1.

32. The method of claim 22, wherein the oligonucleotide further comprises a 2′-O-substituted nucleotide.

33. The method of claim 22, wherein the topoisomerase I inhibitor is CPT-11.

34. A method for inhibiting proliferation of cancer cells comprising:
(a) administering to the cells a first therapeutic agent comprising a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) administering to the cells a second therapeutic agent comprising a topoisomerase I inhibitor,
wherein the administering steps may be performed simultaneously or sequentially in any order.

35. A pharmaceutical composition comprising:
(a) a first therapeutic agent comprising a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) a second therapeutic agent comprising a topoisomerase I inhibitor.

36. A method for treating cancer in an afflicted subject comprising:
(a) administering to the subject a first therapeutic agent comprising a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) administering to the subject a second therapeutic agent comprising a topoisomerase I inhibitor,
wherein the administering steps may be performed simultaneously or sequentially in any order.

37. A method for inhibiting proliferation of cancer cells comprising:
(a) administering to the cells a first therapeutic agent comprising a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) administering to the cells a second therapeutic agent comprising CPT-11,
wherein the administering steps may be performed simultaneously or sequentially in any order.

38. A pharmaceutical composition comprising:
(a) a first therapeutic agent comprising a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) a second therapeutic agent comprising CPT-11.

39. A method for treating cancer in an afflicted subject comprising:
(a) administering to the subject a first therapeutic agent comprising a synthetic, modified oligonucleotide complementary to nucleic acid encoding the N-terminal 8-13 codons of protein kinase A subunit RIα and having from 0 to 25 additional nucleotides extending from the 3′ terminus, the 5′ terminus, or both the 3′ and the 5′ terminus,
wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) administering to the subject a second therapeutic agent comprising CPT-11,
wherein the administering steps may be performed simultaneously or sequentially in any order.

40. A method for inhibiting proliferation of cancer cells comprising:
(a) administering to the cells a first therapeutic agent comprising a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) administering to the cells a second therapeutic agent comprising CPT-11,
wherein the administering steps may be performed simultaneously or sequentially in any order.

41. A pharmaceutical composition comprising:
(a) a first therapeutic agent comprising a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) a second therapeutic agent comprising CPT-11.

42. A method for treating cancer in an afflicted subject comprising:
(a) administering to the subject a first therapeutic agent comprising a synthetic, modified oligonucleotide complementary to at least 15 consecutive nucleotides of the nucleic acid encoding the N-terminal 8-13 codons of protein kinase A subunit RIα, wherein the oligonucleotide is a hybrid, inverted hybrid, or inverted chimeric oligonucleotide,
the hybrid oligonucleotide comprising a region of at least two deoxyribonucleotides, flanked by 3′ and 5′ flanking ribonucleotide regions each having at least four ribonucleotides,
the inverted hybrid oligonucleotide comprising a region of at least four ribonucleotides flanked by 3′ and 5′ flanking deoxyribonucleotide regions of at least two deoxyribonucleotides, and
the inverted chimeric oligonucleotide comprising an oligonucleotide nonionic region of at least four nucleotides flanked by two oligonucleotide phosphorothioate regions; and
(b) administering to the subject a second therapeutic agent comprising CPT-11,
wherein the administering steps may be performed simultaneously or sequentially in any order.

43. A method for inhibiting proliferation of cancer cells comprising:
(a) administering to the cells a first therapeutic agent comprising a synthetic, modified oligonucleotide comprising SEQ ID NO:4, wherein the oligonucleotide has four 2′-O-methyl ribonucleotides at the 3′ terminus and at the 5′ terminus, and wherein the oligonucleotide has phosphorothioate internucleotide linkages between every nucleoside;
and
(b) administering to the cells a second therapeutic agent comprising CPT-11,
wherein the administering steps may be performed simultaneously or sequentially in any order.

44. A pharmaceutical composition comprising:
(a) a first therapeutic agent comprising a synthetic, modified oligonucleotide comprising SEQ ID NO:4, wherein the oligonucleotide has four 2′-O-methyl ribonucleotides at the 3′ terminus and at the 5′ terminus, and wherein the oligonucleotide has phosphorothioate internucleotide linkages between every nucleoside; and
(b) a second therapeutic agent comprising CPT-11.

45. A method for treating cancer in an afflicted subject comprising:
(a) administering to the subject a first therapeutic agent comprising a synthetic, modified oligonucleotide comprising SEQ ID NO:4, wherein the oligonucleotide has four 2′-O-methyl ribonucleotides at the 3′ terminus and at the 5′ terminus, and wherein the oligonucleotide has phosphorothioate internucleotide linkages between every nucleoside;
and
(b) administering to the subject a second therapeutic agent comprising CPT-11,
wherein the administering steps may be performed simultaneously or sequentially in any order.
